SEQUENCE LISTING (32 sequences)

SEQ ID NO: 1 - RNA, 20 nt, synthetic construct
  misc_feature 1..20: note = sgRNA
  source 1..20: mol_type = other RNA
ggttcctacctcccttcctg

SEQ ID NO: 2 - RNA, 20 nt, synthetic construct
  misc_feature 1..20: note = sgRNA
  source 1..20: mol_type = other RNA
tttactggtgctgggaagga

SEQ ID NO: 3 - RNA, 20 nt, synthetic construct
  misc_feature 1..20: note = sgRNA
  source 1..20: mol_type = other RNA
gagtgtgtggagtcctgctc

SEQ ID NO: 4 - RNA, 20 nt, synthetic construct
  misc_feature 1..20: note = sgRNA
  source 1..20: mol_type = other RNA
cagaaaccggcaggctctct

SEQ ID NO: 5 - RNA, 20 nt, synthetic construct
  misc_feature 1..20: note = sgRNA
  source 1..20: mol_type = other RNA
gaggaggggcacagaggaat

SEQ ID NO: 6 - RNA, 20 nt, synthetic construct
  misc_feature 1..20: note = sgRNA
  source 1..20: mol_type = other RNA
ccagtctcaggatggttgag

SEQ ID NO: 7 - RNA, 20 nt, synthetic construct
  misc_feature 1..20: note = sgRNA
  source 1..20: mol_type = other RNA
aggtactagaattaaatgaa

SEQ ID NO: 8 - RNA, 20 nt, synthetic construct
  misc_feature 1..20: note = sgRNA
  source 1..20: mol_type = other RNA
gaaacaggattagaagcctt

SEQ ID NO: 9 - DNA, 20 bp, synthetic construct
  misc_feature 1..20: note = Primer
  source 1..20: mol_type = other DNA
catgggctggcatggtcata

SEQ ID NO: 10 - DNA, 20 bp, synthetic construct
  misc_feature 1..20: note = Primer
  source 1..20: mol_type = other DNA
aacccgatccttgatgccag

SEQ ID NO: 11 - DNA, 20 bp, synthetic construct
  misc_feature 1..20: note = Primer
  source 1..20: mol_type = other DNA
tgtgaaagcagcaaagaggc

SEQ ID NO: 12 - DNA, 23 bp, synthetic construct
  misc_feature 1..23: note = Primer
  source 1..23: mol_type = other DNA
acctcaaactccaaaagaccagt

SEQ ID NO: 13 - DNA, 20 bp, synthetic construct
  misc_feature 1..20: note = Primer
  source 1..20: mol_type = other DNA
gaacctccagtctcagcacc

SEQ ID NO: 14 - DNA, 21 bp, synthetic construct
  misc_feature 1..21: note = Primer
  source 1..21: mol_type = other DNA
gcaggtacagcgtacagttct

SEQ ID NO: 15 - DNA, 20 bp, synthetic construct
  misc_feature 1..20: note = Primer
  source 1..20: mol_type = other DNA
aggatcaagacagcacgtgg

SEQ ID NO: 16 - DNA, 20 bp, synthetic construct
  misc_feature 1..20: note = Primer
  source 1..20: mol_type = other DNA
tactccttgatgtgggcacg

SEQ ID NO: 17 - DNA, 20 bp, synthetic construct
  misc_feature 1..20: note = Primer
  source 1..20: mol_type = other DNA
tgctctcctgttgtgcttct

SEQ ID NO: 18 - DNA, 20 bp, synthetic construct
  misc_feature 1..20: note = Primer
  source 1..20: mol_type = other DNA
agcctcccattcaattgcca

SEQ ID NO: 19 - DNA, 20 bp, synthetic construct
  misc_feature 1..20: note = Primer
  source 1..20: mol_type = other DNA
ggtgactttggtgctaggct

SEQ ID NO: 20 - DNA, 20 bp, synthetic construct
  misc_feature 1..20: note = Primer
  source 1..20: mol_type = other DNA
ggaagacaggagagctgcaa

SEQ ID NO: 21 - DNA, 20 bp, synthetic construct
  misc_feature 1..20: note = Primer
  source 1..20: mol_type = other DNA
cagagagggcaggaggatga

SEQ ID NO: 22 - DNA, 20 bp, synthetic construct
  misc_feature 1..20: note = Primer
  source 1..20: mol_type = other DNA
cctgctgatcactctcgtcg

SEQ ID NO: 23 - DNA, 20 bp, synthetic construct
  misc_feature 1..20: note = Primer
  source 1..20: mol_type = other DNA
gggccttcagagcttgactc

SEQ ID NO: 24 - DNA, 20 bp, synthetic construct
  misc_feature 1..20: note = Primer
  source 1..20: mol_type = other DNA
ttgaacattcggatccgggc

SEQ ID NO: 25 - DNA, 18 bp, synthetic construct
  misc_feature 1..18: note = Primer
  source 1..18: mol_type = other DNA
atggtcaaccccaccgtg

SEQ ID NO: 26 - DNA, 25 bp, synthetic construct
  misc_feature 1..25: note = Primer
  source 1..25: mol_type = other DNA
tttctgctgtctttggaactttgtc

SEQ ID NO: 27 - DNA, 7634 bp, Homo sapiens
  source 1..7634: mol_type = genomic DNA
ctgcaacttccacctctcaggttccagcgattctcctgcctcagcctcccaagtagctaagattacaagcgcccaccaccacgcctggctaatttttgtttttagtagagatggggtttcaccatgttgctcaggctggtcttgaactcctgacctcaagtgatccacccatctcggcctcccaaagcgctgggattacaggcatgagccactgtgccaggcctgcaattacttttgctcctacctaatatcatccccacaaccgccttctgggcagaaaccggcaggctctcttggagaagtcacaggcgtggccatttcctgcaaagagccaaacccccattcctctgtgcccctcctctcccaccaagtgctttataaaaatagctcttgttaccggaaataactgttcatttttcactcctccctcctaggtcacacttttcagaaaaagaatctgcatcctggaaaccagaagaaaaatatgagacggggaatcatcgtgtgatgtgtgtgctgcctttggctgagtgtgtggagtcctgctcaggtgttaggtacagtgtgtttgatcgtggtggcttgaggggaacccgctgttcagagctgtgactgcggtgagtgtttctaaacacccttggtttgggggtagcaaagggcaatggaatggaggctttctcaaacctcacccctgaccccaggactcaggcccagctcatcagggctttgagggaaggttcctacctcccttcctgaggaacaggaaataccctccttcccagcaccagtaaagctgcggtttggagaaacgccagggctagagtgttgtggagaaaccaatcgttgttaacatctcattttcaggctgcactcagagaagctgcccttggctgctcgtagcgccgggccttctctcctcgtcatcatccagagcagccagtgtccgggaggcagaaggtaggctcaagatcagcctggcagaacgccaaacctagggcccctggcacccagaggcgagggggtgcctgctggctgccctgtccccactccctgagctctgttttccactttgttgactaaggtcctccctggggtgggttccggggacaggggaacccaggtccccaagggttcttggttgggtacggctgcacaggacagcttcaagtctgggtctgggatagttgctgccttctttcttcaccacacctgtggtctccctgggtcttggtggggcgtgtatgtgcaggccctgctctgttttcagcaaacctcgctgagacaggagctttggggtgacttattcccagcctgcctcctagaggtgtctctaagagcagcctctgggagtggctgggcaccagggaaaggggaactgggaggaagtgcccagccagagcctcagtcccagaagggcaggagggcaaggggagaatggtcatggatttcttggtgcccacagatgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtgactcactgcagtacccagggacggggtatccaacgtgtgtcactcccttgatgcctagccctgcccctccttgaacctctctggctgagctgggctgggggctggggtctggggtctggctgtcactcacaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaaggtatgacacagggggaggtagaagctctggccaagtggtggctgtggctggtgtgacctgccctgagctgagtactgggagtgggactggtttaaaggctggagtccatggagtagaacctataatgtcctggaacagtgggtttggcaatggcaaaagagggatcaagtcaggagcaggttgggaagccttggaggaggaggaggagttctctgggtgtccttgatggaggccccccagccacatcctgctgtccacagggcctggccccagctgagatctctgcagtgtgtgaaaaagggaatttcaacgtggcccatgggctggcatggtcatattacatcggatatctgcggctgatcctgccaggtgggccatcctctctgcccccatttctcgatctacaaaacagacactaagacacacagcctcttgtgtttctaaacacacagaactcactatatttttcaaagcattttcatgtctaccaaatcctttaatcccaaggcatgaaggatgttgctatgatgcctagtttgcagaaaggaatatggggcttagagccattaagtgacttgcccaaggtcacttgtgaattctttttttttctttttgagaaggaatctccctctgtcgcccaggctagagtgcagtggtaccatctcggctactgcaacctcctccttccaagttcaagtgattatcctggctcaggttcccgagtagctgggattataggcacccaccacaacgcccggctaatttttgtatttttagtagagacagggtttcaccgtattggccaggccggtacaaactcctgacctcaagtgatccgcctgccttagcctcccaaagtgctgggattacaggcgtgagcccagctgtaaattctttcaataaatatttactgcatgctgtgtgctctcatgtgccttttctaattcacaccctatgaggaaggaataatctttattcctgttttattttatttatatattttttaaaaagaagagggttttgctatgttacccagcctggtctcaactcctgggctcaagcaatcctcctgccttggcttcccaaagtgccgagataacaagcaggagccaccaagcccatacctattttagatttttttttttttttttttttgagacaaagtctcactctgttgcgcagactggagtgcagtagcacaatctcagctcactgcgacctctgcctcctgggttcagcctcccaagtagctggaattacaggtccctgccaccggcatggcttttttttttttttttttttttttttttgtatttttagtagagacagggtttcaccatgttgcccaggctggtcttgaattcttgacctcaagtaatccacccaccttggcctcccaaagtgctgggattacagacatgagccactgcaccagccttagatttttttttttttttaagatggagtttcactttttcacccgggctgaagtagagtggcacaatctgggctcactgcaacttctgcttcccaggttccagtgattctcctgccttagcctcccaagtagctgggattttaggtgcctgccaccacgcccggctaatttttgtattttcagtacagacggggtttcaccatgttgggtaggctagtctcaaactcctgacctcaggtgatccacccacctcggcctcccaaagtgctagaactataggcatgagccactgtgcctggcctttttttttaagtgcagttctgagaggtaaagtgatttatccgatatcacatagcttagagagaggaagagaaaggatttgaacccaggtttgtccagagcctggaccctagaccactgcaccgtggagcctgtgtttgttgttgttgttgttgttgttgttgttgttgttgttgttgttgttgttttgagatgaagtctctttctgttacccagcctggagtacagtggtgacaagatctcagctcactgcaacctctgcctcccaggttcaagtgattctcctgcctgagcctcttgaatagctaggattacaggcacgtgccaccatgcctggctaatttttgtatttttagtagagacaatgtttcaccatgttgcccaggctagtctcaaactcctgacctcaaatgatccacctgcctcagcctcccaaagtgctggattacaggcgtgagccactgtgcctgccctaagcctgtgtgttttattcttctgacttgcaggctaaagcggcagctcttccatatctcattgctatctcctagggcttccgctaggagactgatctggggctagaggcctccctctgtgcacacgagaatgctggaaatgtcacctctcagggctctgcctgcctctcagccctgaaagccatggtggaaaggggtggcgctgacatagacatctgaggaaagaagtgagggagggtaaagggtggtgcagtaagaggaggggtggggagggcttttggaggcgctgcccctcctggcctcctgtacaatgagagtgcactggactctccattctctggcacccacacactgcgggggccaatgacctgggtctcactcctgaatcaggtgggagatagggttagcaggaataacttcttgggcttccctgcctcagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccatgctggcatcaaggatcgggtttacagcaacagcatctatgagcttctggagaacgggcagcgggtaagtgtgcaggggagtgggggtctctgaggaggggtcaggaccccagaaccctgggccctagccaagcactgatgaaaattcactgcccttctctgagctgtagtgtccctagctggtcccaggtctgggcaggattcagctctgaatgataatgatgagtaacatttatcgagcacttactacaagctggatgctattctgggtgtgtcatctgatcacctcattgaattctcaccaccaccctatggggtagggactgttagcattcccactttacagaggaggacactgaggctcagagacaccgtaggaaagtggacgatttaaacttgactgtaccatgctcttgaccataaagctgccccagacgaaggctgtgagaacatctgaaggattcatgtgggtgcaggggaacccagaccagagttgaacccagagcctagccccagacctgattcccagctggagcagttgacagcctcgggtcttgccctaggatcttggaagaaggatctgtgaagggtaaatttagcccagccgtgtccctgatttcagagttgggtatcagaggcaaaggcaggccagacagcatagaccccattagggtggccacctcccaggactctatcgttacaggctgagggagtggggcctcagcccgtggcactgggggccagagcctggactggaccctccattctccatccgcctggcccctggcttagtctggtcttcctcttacctcctctaggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccagggtgaggggttggtagggtgaggggttgataggctggagggcaggaggagtcaggggtcccagggtctcccaaagagtcagaaggggctatggagcaccatgccctgggcctgtccttccttccctgggagaagccccctcttcccagcacaccagattgctttttaggtctttgactgtaatccctcaggggagcctgaagaggtgggttctaggggcctctcacttcatctaggattccagaggaaacagaagagtcccaggccagtatgtactgggcacaggagactagggctgcagcctcggctctactactccctcactgtgtgacctcgggcaggtttctgctcctctctgagcattagctttaaaagtaagagggtggaatttcattctctctgaaatcactttttttttttttttttgagatggagtctcactctgttgcccaggctggagtgaagtggctcgatttcgggtcactgcaacctccacctcccgggtccaagcgattctcctgcctcagcctcccaagtagctgggactacaggcgcccgccaccatgcccagctaatttttgtatttttagtagagacggggtttcatcatattggccaggctggtcttgaattcctgacctcatgatctacctgcctcggcctcccaaagtgctaggattacaggcgtgagccactgtgccaggcctgaaccacttctacctctaggattctatgaaactctagtcccagtggctggggcttgggtaggaaggggggtagggagggttaggggatcttcccagcaagttctctaggtatgtttgagtgggaatgggtaagatcctcattactctctcccctatctccctgttccagaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttgagacccagggtcaccaggccagagcctccagtggtctccaagcctctggactgggggctctcttcagtggctgaatgtccagcagagctatttccttccacagggggccttgcagggaagggtccaggacttgacatcttaagatgcgtcttgtccccttgggccagtcatttcccctctctgagcctcggtgtcttcaacctgtgaaatgggatcataatcactgccttacctccctcacggttgttgtgaggactgagtgtgtggaagtttttcataaactttggatgctagtgtacttagggggtgtgccaggtgtctttcatggggccttccagacccactccccacccttctccccttcctttgcccggggacgccgaactctctcaatggtatcaacaggctccttcgccctctggctcctggtcatgttccattattggggagccccagcagaagaatggagaggaggaggaggctgagtttggggtattgaatcccccggctcccaccctgcagcatcaaggttgctatggactctcctgccgggcaactcttgcgtaatcatgactatctctaggattctggcaccacttccttccctggccccttaagcctagctgtgtatcggcacccccaccccactagagtactccctctcacttgcggtttccttatactccacccctttctcaacggtccttttttaaagcacatctcagatta

SEQ ID NO: 28 - DNA, 1167 bp, Homo sapiens
  source 1..1167: mol_type = genomic DNA
ctgcaacttccacctctcaggttccagcgattctcctgcctcagcctcccaagtagctaagattacaagcgcccaccaccacgcctggctaatttttgtttttagtagagatggggtttcaccatgttgctcaggctggtcttgaactcctgacctcaagtgatccacccatctcggcctcccaaagcgctgggattacaggcatgagccactgtgccaggcctgcaattacttttgctcctacctaatatcatccccacaaccgccttctgggcagaaaccggcaggctctcttggagaagtcacaggcgtggccatttcctgcaaagagccaaacccccattcctctgtgcccctcctctcccaccaagtgctttataaaaatagctcttgttaccggaaataactgttcatttttcactcctccctcctaggtcacacttttcagaaaaagaatctgcatcctggaaaccagaagaaaaatatgagacggggaatcatcgtgtgatgtgtgtgctgcctttggctgagtgtgtggagtcctgctcaggtgttaggtacagtgtgtttgatcgtggtggcttgaggggaacccgctgttcagagctgtgactgcggtgagtgtttctaaacacccttggtttgggggtagcaaagggcaatggaatggaggctttctcaaacctcacccctgaccccaggactcaggcccagctcatcagggctttgagggaaggttcctacctcccttcctgaggaacaggaaataccctccttcccagcaccagtaaagctgcggtttggagaaacgccagggctagagtgttgtggagaaaccaatcgttgttaacatctcattttcaggctgcactcagagaagctgcccttggctgctcgtagcgccgggccttctctcctcgtcatcatccagagcagccagtgtccgggaggcagaaggtaggctcaagatcagcctggcagaacgccaaacctagggcccctggcacccagaggcgagggggtgcctgctggctgccctgtccccactccctgagctctgttttccactttgttgactaaggtcctccctggggtgggttccggggacaggggaacccaggtccccaagggttcttggttgggtacggctgcacaggacagcttcaa

SEQ ID NO: 29 - RNA, 6016 nt, synthetic construct
  misc_feature 1..6016: note = dCas9-VPR
  source 1..6016: mol_type = other RNA
aggaaataagagagaaaagaagagtaagaagaaatatagctagcgccaccatggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgataaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttgagcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaa

SEQ ID NO: 30 - RNA, 4116 nt, synthetic construct
  misc_feature 1..4116: note = dCas9
  source 1..4116: mol_type = other RNA
atggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgataaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgac

SEQ ID NO: 31 - RNA, 1569 nt, synthetic construct
  misc_feature 1..1569: note = Fusionprotein of VP64, p65 and RTA
  source 1..1569: mol_type = other RNA
gacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttt

SEQ ID NO: 32 - RNA, 80 nt, Streptococcus pyogenes
  source 1..80: mol_type = other RNA
gttttagagctagaaatagcaagttaaaataaggctagtccgttatcaacttgaaaaagtggcaccgagtcggtgctttt

(19) United States
(12) Patent Application Publication
(10) Pub. No.: US 2026/0015599 A1
(43) Pub. Date: Jan. 15, 2026

(54) TARGETING THE STING1 GENE BY CRISPR ACTIVATION

(71) Applicant: AARHUS UNIVERSITET, Aarhus C (DK)

(72) Inventors: Stine Høvring GODSK, Aarhus C (DK); Kristine Raaby GAMMELGAARD, Aarhus C (DK); Martin Roelsgaard JAKOBSEN, Aarhus C (DK); Rasmus Otkjær BAK, Aarhus C (DK); Anders ETZERODT, Aarhus C (DK)

(21) Appl. No.: 18/874,333

(22) PCT Filed: Jun. 1, 2023
(86) PCT No.: PCT/EP2023/064710

§ 371 Date: Dec. 12, 2024

(30) Foreign Application Priority Data
Jun. 20, 2022 (EP) 22179901.8

(51) Int. Cl. C12N 9/22; A61K 38/00; C12N 15/113

(52) U.S. Cl. CPC C12N 9/226; C12N 15/1138; A61K 38/00; C12N 2310/20

(57) ABSTRACT

The present invention relates to CRISPRa constructs targeting STING for use in cancer treatment. In particular, the present invention relates to CRISPRa constructs targeting STING for use in treatment of cancers having no or low STING expression.

### TECHNICAL FIELD OF THE INVENTION

The present invention relates to epigenetic regulation of the STING gene (a.k.a. STING1 and TMEM173) using CRISPR activation (CRISPRa) constructs targeting a region surrounding the transcriptional start site of the human STING gene for use in immunotherapy concepts. In particular, the present invention relates to CRISPRa constructs targeting STING for use in treatment of cancers having no or low STING expression.

### BACKGROUND OF THE INVENTION

Anti-cancer immune responses within tumors are essential for controlling cancer development or elimination. Today, treatments that that has demonstrated the ability to gear-up the immune system for improved anti-tumor immune responses are approved at a high pace. Despite significant advances, only a minority of patients respond well to these new treatment options defined in broad terms as “immunotherapies”.

STING is a recently discovered protein with pleiotropic effects in the immune system. It is part of the cGAS-STING pathway which is the central cellular cytosolic double-stranded DNA (dsDNA) sensor system. The cGAS-STING pathway allows the innate immune system to respond to infections, inflammation, and cancer Understanding how intrinsic and extrinsic self-DNA sensing contribute to STING pathway activation is highly relevant in order to understand cancer biology. For example, in cancer cells DNA instability and deficiencies in DNA repair systems are major causes of DNA accumulation and activation of the cGAS-STING pathway. Upon sensing of dsDNA, cGAS catalyzes the formation of 2′,3′-cyclic GMP-AMP (cGAMP), which activates STING and initiates a cascade of cellular events that ultimately support transcriptional induction of immune-stimulated genes, inflammatory cytokines, and type I interferon (IFN). These factors participate in altered tumor tissue architecture, increased infiltration of immune cells into the tumor, and activation of a broad range of immune functions.

Preclinical studies focusing on activating the STING pathway have shown that this pathway contributes to mounting an efficient anti-tumor response, mediated through T-cell activation and increased presentation of tumor antigens by dendritic cells (DCs). The involvement of tumor-infiltrating myeloid cells, such as macrophages and DCs, in both the activation and the suppression of T-cell driven anti-tumor immunity is also well documented. Importantly, multiple recent preclinical and clinical data demonstrate that proper activation of the innate immune system in the tumor microenvironment (TME) is involved in orchestrating the necessary level of adaptive anti-tumor immunity.

Studies in murine cancer models have shown limited effect of immunotherapy in absence of STING, suggesting that early innate immune responses and endogenous STING activation within tumors is essential for anti-tumor effects. Furthermore, combining different types of immunotherapies with STING agonist therapy increases the effect of tumor control.

Many cancers have been proved to epigenetically downregulate STING expression as part of an immune escape mechanism. This will deductively result in a STING agonist, meant to activate an already present protein, to be useless and non-functional. Thus, clinical evidence demonstrating anti-tumor responses using STING agonist treatment is sparse.

Botto Sara et al (MBIO, vol. 10, no. 1, 26 Feb. 2019) describes cells treated with a CRISPR-Cas9 targeting STING.

Origene Technologies Inc.: “Product datasheet for GA117456 discloses a CRISPRa construct targeting the TMEM173 (STING) gene and composed of a dCas9 protein associated with VP64 (transcription activator) and a gRNA.

Falahat Rana et al. (Proceedings of the National Academy of Sciences, vol. 118, no. 15, 7 Apr. 2021) describes a composition for re-activation of STING in order to re-sensitize cancer cells.

WO 2019/204503 A1 shows examples of CRISPRa constructs, comprising a dCas9, gRNA targeted the target gene and a transcriptional activator fused with dCas9, the constructs being useful for treatment of cancer. Further disclosed is numerous sgRNAs intended for targeting specific genes, including an sgRNA targeting TMEM 173.

Hence, an improved method of exploiting the regulation and activation of the cGAS-STING pathway in cancer treatment would be advantageous.

### SUMMARY OF THE INVENTION

Epigenetic regulation has emerged as a key mechanism for how cancer cells adapt to the host immune system. This can increase cancer cell fitness by influence gene expression without the creation of permanent changes to the genome. Thus, treatment involving epigenetic modulation (epi-drugs) has become an attractive strategy for cancer therapy, particularly combination therapies with conventional therapies such as chemotherapy have shown beneficial effects compared with each treatment alone. Unfortunately, off-target effects have so far been a problematic issue for epi-drugs.

The present invention is based on the identification of STING expression being downregulated in certain cancers and exploits this fact by using a CRISPRa approach targeting STING selectively. The result is a reactivation of endogenous STING expression and the immunologically activation of cancer cells.

Selected overview of the data is presented in the following:
Examples 1-2 demonstrate that STING expression can be found genetically suppressed in some human cancer cell lines.
Examples 4-6 demonstrate that human cancer cells can be genetically re-programmed to transiently express STING using CRISPRa.
Example 7 demonstrates that STING re-expression using CRISPRa can prone human cancer cells to respond to STING activation and become immunologically active.
Example 8 demonstrates that STING re-expression using CRISPRa can prone human cancer cells to respond to STING activation and become immunologically active following treatment with chemotherapy drugs.
Examples 10-12 verifies the human data in various murine models.
Example 13 demonstrates that STING re-expression using a combination of sgRNA and dCas9-VPR mRNA can activate the STING pathway to respond to endogenous levels of cGAMP within cells, which is produced as a consequence of DNA instability and/or increased micronuclei formation leading to DNA cytosolic accumulation and activation of cGAS.
Example 14 demonstrates that re-expression of STING in cancer cells can be achieved by delivery of sgRNA and dCas9 mRNA by lipid nanoparticles.

Thus, an object of the present invention relates to re-activation of STING expression in cancer cells with low or no endogeneous STING expression, thereby overcoming the immunological escape of the cancer cells and revive an immune response in the tumor.

In particular, it is an object of the present invention to provide a treatment that solves the above-mentioned problems of the prior art with low or no STING activity in some cancer cells.

Thus, one aspect of the invention relates to a composition or combination comprising
a)
   an RNA molecule encoding an RNA-guided protein, such as a deactivated RNA-guided endonuclease, such as dCas, preferably dCas9; or
   an RNA-guided protein, such as a deactivated RNA-guided endonuclease, such as dCas, preferably dCas9; and
b) a guide RNA complementary to a part of STING DNA (preferably within SEQ ID NO: 28 or within the complementary sequence of SEQ ID NO: 28); and
c) a transcriptional activator, preferably
   encoded by the RNA molecule encoding the RNA-guided protein; or
   fused to the RNA-guided protein.

An aspect of the invention also relates to a CRISPR activation (CRISPRa) composition or a CRISPRa combination comprising
a)
   an RNA molecule encoding a deactivated RNA-guided endonuclease, such as dCas, preferably dCas9; or
   a deactivated RNA-guided endonuclease, such as dCas, preferably dCas9; and
b) a guide RNA complementary to a part of STING DNA; and
c) a transcriptional activator
   encoded by the RNA molecule encoding the deactivated RNA-guided endonuclease (as a fusion construct); or
   fused to the deactivated RNA-guided endonuclease;
      wherein the guide RNA binds to STING DNA within SEQ ID NO: 28 or the complementary sequence of SEQ ID NO: 28, such as within position 200-900 of SEQ ID NO: 28 or the complementary sequence of SEQ ID NO: 28, preferably within position 240-840, and more preferably within position 240-420 and even more preferably within position 260-380 and most preferably within position 270-300 of SEQ ID NO: 28 or the complementary sequence of SEQ ID NO: 28.

Yet an aspect of the invention relates to a kit or combination comprising
a) a composition or combination according to the invention; and
b) a STING stimulatory agent.

Another aspect of the present invention relates to the composition or combination or kit according to the invention for use as a medicament.

In particular, the compositions, combinations, and kits according to the invention have medical uses in relation to cancer. Thus, an aspect of the invention relates to the composition or combination or kit according to the invention, for use in the treatment or alleviation of cancer.

Yet another aspect of the present invention is to provide the composition or combination or kit according to the invention, for use in the treatment or alleviation of cancer.

Still another aspect of the present invention is to provide an isolated guide RNA comprising a sequence of at least 15 nucleotides, preferably 19-21 nucleotides such as 20 nucleotides, which is complementary STING DNA within SEQ ID NO: 28 or the complementary sequence of SEQ ID NO: 28, such as within position 200-900 of SEQ ID NO: 28 or the complementary sequence of SEQ ID NO: 28, preferably within position 240-840, and more preferably within position 240-420 and even more preferably within position 260-380 and most preferably within position 270-300 of SEQ ID NO: 28 or the complementary sequence of SEQ ID NO: 28.

### BRIEF DESCRIPTION OF THE FIGURES

FIG. 1 shows the expression levels (protein and mRNA) of target genes involved in regulating the cGAS-STING pathway from four different human sarcoma cell lines. The data demonstrates that STING expression is genetically suppressed in some cancer cell lines but importantly not all.

FIG. 2 shows the expression of cytokines (IFNb, CXCL10, IL6, CCL5) in four different human sarcoma cell lines stimulated with HT-DNA (a ligand for cGAS that facilitates cGAS activation, which in turn produces endogenous 2′3′-cGAMP) or with a synthetically generated STING agonist, 2′3′-cGAMP. An agonist not related to the cGAS-STING pathway (Poly (I:C)) was used as control. The data demonstrates that genetically suppressed STING expression affects the ability of cancer cells to create an immunological response to agonists specifically targeting cGAS and STING.

FIG. 3 shows a conceptual illustration of STING transcriptional activation using the CRISPRa approach.

FIG. 4 shows the degree of STING epigenetic reprogramming in a human sarcoma cell line following CRISPRa with individual and combined sgRNAs targeting the region surrounding the transcriptional start site of the human STING gene. The data demonstrates that STING expression can be transiently induced (both mRNA and protein) using several individual sgRNAs combined with dCas9-VPR expression within cancer cells. Importantly, the design of one single sgRNA is superior to the effect of using many different sgRNAs in combination.

FIG. 5 shows the kinetics of STING protein expression following epi-genetic reprogramming using the CRISPRa approach. Two human sarcoma cell lines was used whereof one cell line has no STING expression and the other expresses STING. The data demonstrates that STING can be transiently over-expressed using several individual sgRNAs combined with dCas9-VPR expression within cancer cells with both no STING expression and normal endogenous STING expression.

FIG. 6 shows the kinetics of STING mRNA expression from the same experiment as in FIG. 5.

FIG. 7 shows various cytokine protein expression levels in a human sarcoma cell line following epigenetic reprogramming of STING using the CRISPRa approach (the condition where cells were subjected to CRISPRa are designated with STING+ in superscript). The cells were subsequently stimulated with HT-DNA (FIG. 7A-C) or cGAMP (FIG. 7D-F) to trigger cGAS-STING activation. The data demonstrates that STING re-expression obtained using a combination of a specific sgRNA and dCas9-VPR mRNA can make cancer cells immunologically active by secreting cytokines in response to STING activation.

FIG. 8 shows various cytokine protein expression levels in a human sarcoma cell line following epi-genetic reprogramming of STING using the CRISPRa approach (the condition where cells were subjected to CRISPRa are designated with STING+ in superscript). The cells were subsequently stimulated with chemotherapy to trigger DNA damage and thereby lead to cGAS-STING activation. The data demonstrates that STING re-expression obtained using a combination of a specific sgRNA and dCas9-VPR mRNA can make cancer cells immunologically active by secreting cytokines in response to treatment with chemotherapeutic drugs.

FIG. 9 shows endogenous STING expression in various human and murine cancer cell lines.

FIG. 10 shows the degree of STING epigenetic reprogramming in a murine lung cancer cell line following CRISPRa with individual sgRNAs as well as pooled sgRNAs targeting a region surrounding the transcriptional start site of murine STING. The data demonstrates transient STING expression (both mRNA and protein) resulting from the use of several individual sgRNAs combined with dCas9-VPR in the cancer cells.

FIG. 11 shows the kinetics of both STING protein expression and STING mRNA expression in two murine cancer cell lines (a triple-negative breast cancer and lung cancer cell line) following epigenetic reprogramming using the CRISPR/Cas9 activation approach.

FIG. 12 shows the protein expression levels of various cytokines in murine cancer cell lines that were subjected to STING epigenetic reprogramming using CRISPRa and subsequently stimulated with HT-DNA to trigger cGAS-STING activation (the condition where cells were subjected to CRISPRa are designated with STING+ in superscript). The data demonstrates that STING re-expression obtained using a combination of a specific sgRNA and dCas9-VPR mRNA can make cancer cells immunologically active by secreting cytokines in response to STING activation.

FIG. 13 shows the immunological effects of STING epigenetic reprogramming using CRISPRa and subsequently activation due to cancer cells endogenous DNA sensing and cGAMP production. In the triple-negative cancer cell line 41T, which have epigenetic silenced STING, endogenous 2′3′-cGAMP production can be detected both intracellular and extracellular (FIG. 13A). This was not apparent in another cancer line, YUMM1.G, who has high endogenous STING expression. The production of cGAMP correlated with a higher degree of micronuclei formation per cell, a known source for DNA sensing by cGAS (FIG. 13B). The data finally demonstrate that STING re-expression in cells with endogenous cGAMP production results in a time-dependent STING activation (FIG. 13C—phosphorylated STING signal) and downstream induction of interferon-stimulatory genes (FIG. 13C—ISG15 and STAT1).

FIG. 14 shows the differential expressed genes in 4T1 cells after CRISPRa of STING. RNaseq transcriptomic analysis identified a large group of highly upregulated genes that peaked at day 3 post CRISPRa STING treatment. No genes were significantly downregulated. Exploration of STING mRNA expression demonstrated that this peaked at day 1 and reached background levels at day 4. This suggest that STING mRNA is rapidly induced and the subsequent days of accumulation of STING protein reactivated an IFN signaling pathway supporting a broad gene induction signature in the cancer cells.

FIG. 15 shows CRISPRa STING reactivation within 4T1 cells 48 hours after RNA components were delivered by lipid nano particles (LNPs). A 3×3 matrix of concentrations (500-2000 ng) of dCas9 mRNA as well as sgRNA was evaluated. The data demonstrate a linear correlation between the two components in regard to STING protein reexpression.

FIG. 16 shows gene expression of STING, IFNb and CXCL10, within 4T1 cells over 6 days following CRISPRa STING conducted by LNP RNA delivery. The data demonstrate that CXCL10 (FIG. 16A) and IFNb (FIG. 16B) gene induction was measurable 2 days after LNP treatment and peaked at 3 day. In contrary, STING gene expression was induced already at day 1, but peaked also at day 3, following a rapid decline at day 4 (FIG. 16C).

FIG. 17 shows a schematic presentation of the binding sites for sgRNA #1 to #5 in the human STING gene. The black bar annotated as ‘STING’ indicates the DNA-sequence of the transcribed region. Exon 1 and Exon 2 are indicated in gray bars. Binding sites for CRISPRa sgRNAs #1-5 are annotated as arrows. Right-pointing arrows indicate a protospacer and PAM on the bottom strand and left-pointing arrows indicate a protospacer sequence and PAM on the top strand.

The present invention will be described more detailed in the following.

### DETAILED DESCRIPTION OF THE INVENTION

### Definitions

Prior to discussing the present invention in further details, the following terms and conventions will first be defined:

### CRISPR-Cas

The CRISPR-Cas system relies on two main components: a guide RNA (gRNA/sgRNA) and a CRISPR-associated (Cas) nuclease.

The guide RNA (sgRNA or gRNA) is a specific RNA sequence that recognizes the target DNA region of interest and directs the (Cas) nuclease there for editing. The gRNA is made up of two parts: crispr RNA (crRNA), of which a 17-20 nucleotide sequence is complementary to the target DNA; and a tracr RNA, which serves as a binding scaffold for the nuclease. In the present context, sgRNA and gRNA are used interchangeably.

The CRISPR-associated protein is a non-specific endonuclease. It is directed to the specific DNA locus by a gRNA, where it makes a double-strand break. There are several versions of Cas nucleases isolated from different bacteria. The most commonly used one is the Cas9 nuclease from Streptococcus pyogenes, which is also used in the example section.

The guide RNA (gRNA/sgRNA) contains at least two parts, i) the “spacer” or “DNA binding part” most often consisting of ˜20 nucleotides; and ii) the “scaffold sequence” or “nuclease-recruiting sequence (tracrRNA)” necessary for Cas-binding. SEQ ID NO 32 is the scaffold sequence used in the example section.

### CRISPRa

“CRISPR activation” or “CRISPRa” is a variant of CRISPR in which a catalytically dead or deactivated Cas protein (dCas), such as Cas9, is fused with one or more transcription factors. dCas nuclease is opposed to Cas nuclease unable to exert its nuclease ability and thus cannot make double strand breaks. The guide RNA navigates the dCas to the genome along with the transcription factors, which will initiate transcription of the target gene.

In the present context the terms “dead Cas protein”, “deactivated Cas protein” and “dCas” (such as “dCas9”) are used interchangeably.

### Compositions and Combinations

As outlined above, the present invention utilizes the knowledge of STING expression being downregulated in several cancers. By using a CRISPR activation (CRISPRa) approach specifically targeting STING, endogenous STING expression can be reactivated and thereby make cancer cells immunologically active. Thus, an aspect of the invention relates to a composition or combination comprising
a)
   an RNA molecule encoding for RNA-guided proteinaceous molecule, such as a deactivated RNA-guided endonuclease, such as dCas, preferably dCas9; or
   an RNA-guided proteinaceous molecule, such as a deactivated RNA-guided endonuclease, such as dCas, preferably dCas9; and
b) a guide RNA complementary to a part of STING DNA (preferably within SEQ ID NO: 28 or within the complementary sequence of SEQ ID NO: 28); and
c) a transcriptional activator, preferably
   encoded by the RNA molecule encoding for the RNA-guided proteinaceous molecule; or
   fused to the RNA-guided proteinaceous molecule.

An aspect of the invention also relates to a CRISPR activation (CRISPRa) composition or a CRISPRa combination comprising
a)
   an RNA molecule encoding a deactivated RNA-guided endonuclease, such as dCas, preferably dCas9; or
   a deactivated RNA-guided endonuclease, such as dCas, preferably dCas9; and
b) a guide RNA complementary to a part of STING DNA; and
c) a transcriptional activator
   encoded by the RNA molecule encoding the deactivated RNA-guided endonuclease (as a fusion construct); or
   fused to the deactivated RNA-guided endonuclease;
      wherein the guide RNA binds and direct deactivated RNA-guided endonuclease such as dCas9 to STING DNA sequences within SEQ ID NO: 28 or the complementary sequence of SEQ ID NO: 28, such as within position 200-900 of SEQ ID NO: 28 or the complementary sequence of SEQ ID NO: 28, preferably within position 240-840, and more preferably within position 240-420 and even more preferably within position 260-380 and most preferably within position 270-300 of SEQ ID NO: 28 or the complementary sequence of SEQ ID NO: 28.

It is to be understood that when the transcriptional activator is encoded by the RNA molecule encoding the deactivated RNA-guided endonuclease, such that they will be expressed as a fused construct giving rise to the transcriptional activator fused to the deactivated RNA-guided endonuclease. Hence, in a preferred embodiment, the transcriptional activator and the deactivated RNA-guided endonuclease are encoded as a fusion protein by the RNA molecule. Phrased in another way, the transcriptional activator and the deactivated RNA-guided endonuclease are operably linked in the RNA molecule.

In a preferred embodiment, the composition or combination comprises an RNA molecule encoding for RNA-guided proteinaceous molecule, such as encoding for dCas9. In the example section, RNA molecules encoding dCas9 has been used, exemplified by SEQ ID NO: 30 (without a transcriptional activator) and SEQ ID NO: 29 (with transcriptional activator sequence (VPR)).

In an alternative embodiment, the composition or combination comprises an RNA-guided proteinaceous molecule, such as a deactivated RNA-guided endonuclease. Thus, a protein version of e.g. dCas9 may also be used.

In yet a preferred embodiment, the composition or combination is a CRISPRa construct.

The guide may bind different regions in the STING DNA. Thus, in an embodiment, the guide RNA is complementary to STING DNA within the promoter-region of STING. In another embodiment, the guide RNA is complementary to STING DNA in the vicinity of the transcriptional start site of STING, such as within 500 nucleotides upstream or downstream from the transcriptional start site, preferably within 400 nucleotides upstream of the transcriptional start site, more preferably 400 to 50 nucleotides upstream of the transcriptional start site.

For some CRISPR/Cas systems, there is only one guide RNA molecule called a CRISPR RNA (crRNA). For others like Cas9, there are two molecules that hybridize to make up the guide RNA (called tracrRNA and crRNA, respectively). In many applications, these two small RNAs have been fused into a single RNA strand, which is called a single guide RNA (sgRNA). In here sgRNAs has been used, but the two-part system can also work quite effectively and are more cost efficient to produce.

Thus, in an embodiment, the guide RNA comprises a crRNA, a tracrRNA and a crRNA, or an sgRNA.

In yet an embodiment, the guide binds to STING DNA within SEQ ID NO: 28 or the complementary sequence of SEQ ID NO: 28, such as within position 200-900 of SEQ ID NO: 28 or its complementary sequence, preferably within position 240-840, and more preferably within position 240-420 and even more preferably within position 260-380 and most preferably within position 270-300 of SEQ ID NO: 28 or its complementary sequence.

It is to be understood that the guide RNA (such as sgRNA) may bind either of the strands of the double stranded genomic STING DNA, preferably as outlined in the example section. Thus, in an embodiment, the guide RNA binds to (is complementary to) either the “+”-strand or the “−”-strand of STING DNA, preferably within SEQ ID NO: 28 or its complementary sequence.

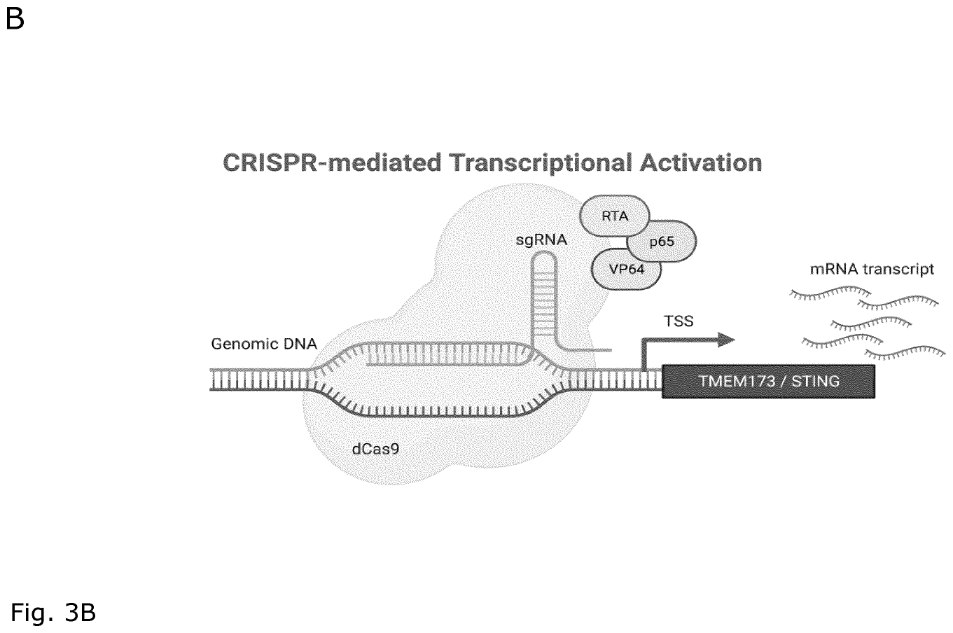

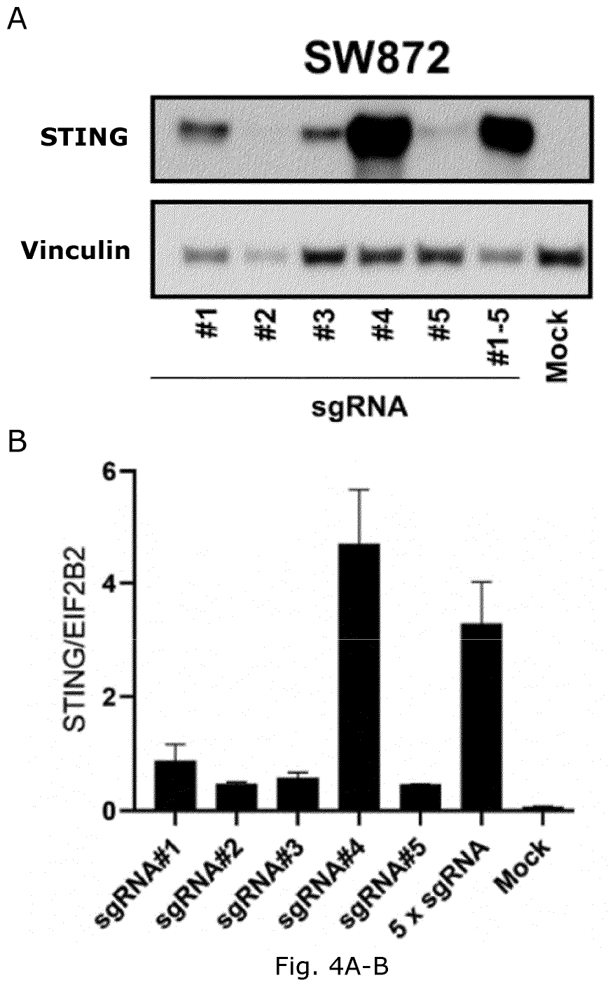

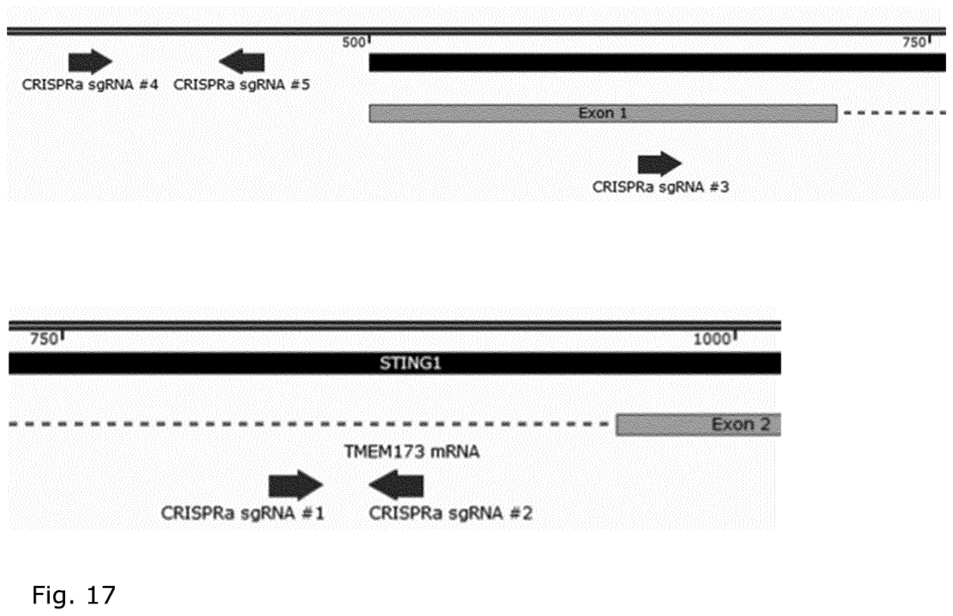

As shown in example 4 (see also FIG. 4) different sgRNAs binding within SEQ ID NO: 28 or its complementary sequence have been tested showing that the sgRNA may bind different regions and strands while still being able to activate expression of STING. SEQ ID NO: 4 showed the highest activity. See also FIG. 17 showing binding positions and to which strand within SEQ ID NO: 28.

In an embodiment, the DNA binding part of the guide RNA has a length of 18-25 nucleotides, preferably 19-21 nucleotides and most preferably 20 nucleotides. In the example section the DNA binding part is 20 nucleotides.

Guide RNAs comprise a DNA binding part and a protein (dCas9) binding part. Thus, in yet an embodiment, the guide RNA (sgRNA) has a complete length in the range 50-150 nucleotides, such as 80-120, or such as 90-110.

In a preferred embodiment, the guide RNA (such as sgRNA) comprises one or more oligonucleotide sequences identical or complementary to sequences selected from the group consisting of SEQ ID NO's: 1-8, preferably selected from SEQ ID NOs: 1-5, more preferably SEQ ID NO: 4. As outlined above, the guide RNA may bind to either of the strands in the genomic DNA. Both strands have been tested in the example section and also outlined in Table 2 and FIG. 17.

It is well-known that guide RNAs may comprise one or more modified or nucleotides e.g. to increase hybridization or increase stability in vitro and in vivo. Thus, in an embodiment, the guide RNA comprises one or more modified nucleotides.

In another embodiment, the one or more modified nucleotides is selected from the group consisting of Bridged nucleic acids (BNAs), Locked nucleic acids (LNA), sugar-modified nucleotides and 2-Methyl-RNA.

In yet an embodiment, the artificial nucleotides are positioned in one or more of position 1-5 in the 5′-end and/or 3′-end of the guide RNA, preferably in position 1-5 of both the 5′-end and/or 3′-end of the guide RNA, such as position 1-4 of both the 5′-end and/or 3′-end of the guide RNA, such as position 1-3 of both the 5′-end and/or 3′-end of the guide RNA, such as position 1-2 of both the 5′-end and/or 3′-end of the guide RNA, such as position 1 of both the 5′-end and/or 3′-end of the guide RNA. As outlined in the example section (see Example 4), the tested guide RNAs (sgRNAs) comprise three 2′-O-methyl 3′phosphorothioate modified nucleotides at each end.

In an embodiment, at least one modified sugar moiety is a 2′-substituted sugar moiety. In an embodiment, said 2′-substituted sugar moiety has a 2′-substitution selected from the group consisting of 2′-O-Methyl (2′-OMe), 2′-fluoro (2′-F) and 2′-O-methoxyethyl (2′-MOE).

In an embodiment, said 2′-substitution of said at least one 2′-substituted sugar moiety is a 2′-O-methoxyethyl (2′-MOE).

In another embodiment the artificial nucleotide is 2′-O-methyl 3′phosphorothioate.

In an embodiment, at least one modified sugar moiety is a bicyclic sugar moiety.

In an embodiment, at least one bicyclic sugar moiety is a locked nucleic acid (LNA) or constrained ethyl (cEt) nucleoside.

In an embodiment, at least one sugar moiety is a sugar surrogate.

In an embodiment, at least one sugar surrogate is a morpholino.

In an embodiment, at least one morpholino is a modified morpholino.

In an embodiment, the guide RNA comprises at least one internucleoside N3′ to P5′ phosphoramidate diester linkage.

In an embodiment, the modified oligonucleotide comprises at least one internucleoside phosphorothioate linkage.

In an embodiment, all internucleoside linkages are phosphorothioate.

As outlined in the example section the invention is based on CRISPRa using inactivated endonucleases. Thus, in an embodiment, the RNA molecule encoding the RNA-guided protein, encodes a deactivated RNA-guided endonuclease or the RNA-guided protein is a deactivated RNA-guided endonuclease, such as a dCas, preferably a dCas9.

In yet an embodiment, the deactivated RNA-guided endonuclease is a dCas, such as from Streptococcus pyogenes (SpCas9), from Staphylococcus aureus (SaCas9), Cas12a from Lachnospiraceae sp (LbCas12a), Cas12a from Acidaminococcus sp. (AsCas12a), and Cas12f1 from Acidibacillus sulfuroxidans (AsCas12f), preferably dCas9 and more preferably Sp-dCas9 (used in the example section). All of these Cas9 proteins listed have been used in CRISPRa systems.

Different transcriptional activators may be used in CRISPRa systems. Thus, in an embodiment, the transcriptional activator is selected from the group consisting of RTA, p65, VP16, HSF1, MyoD1, VP64, VP160 (VP64 and VP160 are 4 and 10 repeats of the activator VP16), repeats of VP16, such as 2-16 repeats, CBP, p300, and combinations thereof, preferably being a combination of VP64, p65, and RTA (a.k.a. VPR). In the example section, VPR (a combination of RTA, p65, and VP64) have been used (SEQ ID NO: 31 (known as VPR)).

In an embodiment, dCas is fused to a scaffold that recruits activator peptides, such as SunTag.

In another embodiment, dCas is fused to a series of activation domains, such as dCas9-VPR; as used in the example section.

In yet another embodiment, dCas is fused to an activator and a tagged gRNA recruits other activators, such as SAM.

The transcriptional activators may be coupled in different ways. Thus, in an embodiment, the transcriptional activator is fused to the RNA-guided protein, such as to a deactivated RNA-guided endonuclease, preferably dCas9.

In another embodiment, the transcriptional activator is encoded by the RNA encoding the RNA-guided protein, such that the RNA-guided protein and transcriptional activator are operatively linked.

In yet another embodiment, the transcriptional activator is coupled to the guide RNA, with the provision that part of the transcriptional activator binds to RNA, such as through interactions with MS2. SAM uses specially engineered guide RNAs to increase transcription. This is done through creating a dCas9-VP64 fusion protein and a sgRNA carrying MS2 hairpin aptamers that bind to MS2 binding proteins. These MS2 binding proteins are fused to additional activation domains (HS1 and p65).

The SunTag activator system uses the dCas9 protein, which is modified to be linked with the SunTag. The SunTag is a repeating polypeptide array that can recruit multiple copies of antibodies. Through attaching transcriptional factors on the antibodies, the SunTag dCas9 activating complex amplifies its recruitment of transcriptional factors. In order to guide the dCas9 protein to its target gene, the dCas9 SunTag system uses sgRNA.

In a further embodiment, the transcription activator is placed/fused in a spatial orientation, which allows it to affect the transcription of the STING gene.

To improve in vivo uptake, the composition or combination may be located in lipid nanoparticles (LNPs). Thus, in an embodiment, the composition or combination according to the invention is located in/on LNPs. In an embodiment, the RNA molecule encoding an RNA-guided protein or the RNA-guided protein and the guide RNA comprising a sequence complementary to a part of STING DNA (such as SEQ ID NO: 28 or its complementary sequence) may be positioned in the same LNP.

In yet an embodiment,
the RNA molecule encoding an RNA-guided protein or the RNA-guided protein is positioned in a first LNP; and
the guide RNA comprising a sequence complementary to a part of STING DNA (such as SEQ ID NO: 28 or its complementary sequence) is positioned in a second LNP.

It may be advantageous if the LNP's were able to target the LNPs to a place of interest in vivo (such as cancer cells). Thus, in an embodiment, LNPs comprises a cancer targeting moiety on the surface, such as an antibody or a peptide or a ligand or a chemical structure.

As mentioned above, the different components may be located in different LNPs. To improve specificity of the method, each type of LNP may comprise different targeting moieties. Thus, in an embodiment for the CRISPRa composition or CRISPRa combination according to the invention,
the first LNP comprises a first cancer targeting moiety on the surface, such as an antibody or a peptide or a ligand or a chemical structure; and
the second LNP comprises a second cancer targeting moiety on the surface, such as an antibody or a peptide or a ligand or a chemical structure;
   wherein the first and the second cancer targeting moiety target different cell surface proteins on the cancer.

In an embodiment, the CRISPRa composition or CRISPRa combination according the invention has cancer targeting moieties having affinity for one or more cancer targets selected from the group consisting of EpCAM, HER2, CD70, CD33, GD2, MAGE-A4, MSLN, PSMA, EGFR, CLDN18, CLDN1, CLDN6, and MUC1.

### Kit of Parts

The composition or combination according to the invention may form part of a kit. Thus, an aspect of the invention relates to a kit or combination comprising
a) a composition or combination according to the invention; and
b) a STING stimulatory agent.

In an embodiment, the STING stimulatory agent is selected from the group consisting of
nucleic acids, such as cyclic-di-nucleotide (CDNs), such as cyclic 2′3′ GMP-AMP (cGAMP), such as chemical stabilized forms of 2′3′ cGAMP;
enzymatic resistant double stranded DNA supporting intracellular cGAS activity;
DNA damage repair (DDR) inhibitors including but not limited to: PARP inhibitors; ATM inhibitors, Topoisomerase inhibitors, DNA crosslinking agents, microtubule-targeting drugs, or antimetabolites;
Small molecules capable of binding to and activating STING;
Checkpoint inhibitors; and/or
ENPP1 inhibitors.

Thus, the composition or combination according to the invention activates STING expression and the STING stimulatory agent activates STING activity.

### Medical Claims

The compositions, combinations, and kits according to the invention have medical uses, such as in relation to cancer treatment. Thus, an aspect of the invention relates to the composition or combination or kit according to the invention for use as a medicament.

In particular, the compositions, combinations, and kits according to the invention have medical uses in relation to cancer. Thus, an aspect of the invention relates to the composition or combination or kit according to the invention, for use in the treatment or alleviation of cancer. For example, Examples 7-8 demonstrate that STING re-expression using CRISPRa can sensitize human cancer cells to respond to STING activation and become immunological active following treatment with chemotherapy drugs.

In an embodiment, the cancer is associated with no or low STING activity, such as insufficient STING activity, increased chromosomal instability and/or elevated cGAMP production.

The invention may find use in relation to different cancer types. Thus, in an embodiment, said cancer is a solid cancer, such as selected from the group consisting of, brain cancer, glioblastoma, lung cancer, colorectal cancer, skin cancer, pancreas cancer, bladder cancer, liver cancer, breast cancer, eye cancer and prostate cancer.

In a further embodiment, the cancer is a metastatic cancer, a refractory cancer (e.g., a cancer refractory to previous cancer therapy), and/or recurrent cancers.

In yet a further embodiment, said cancer is a haematological cancer, such as selected from the group consisting of multiple myeloma, acute myeloblastic leukemia, chronic myelogenic leukemia, acute lymphoblastic leukemia and chronic lymphocytic leukemia.

In another embodiment, the compositions, combinations and kits according to the invention is for treatment or amelioration of a subject who has undergone cancer therapy or is undergoing cancer therapy, or who is scheduled for cancer therapy of said cancer with a different anti-cancer therapy, such as with immunotherapy, chemotherapeutics and/or by radiation.

In a related embodiment, said therapy is selected from the group consisting of immunotherapy, radiation therapy and therapy by chemotherapeutics.

An effect of cancer therapy is killing of cancer cells, which releases DNA (and other cellular components) to the nearby in vivo environment (and to the blood stream) and therefore act as the STING stimulatory agent. Thus, without being bound by theory, compositions, combination and/or kits according to the invention, could be administered simultaneously with the cancer therapy or shortly after the cancer therapy, such as, but limited to, within 48 hours or within 24 hours from initiation of the cancer therapy session.

In an embodiment, the chemotherapeutic (anti-cancer drug) is selected from the group consisting of
DNA damage repair (DDR) inhibitors including but not limited to: PARP inhibitors;
ATM inhibitors, Topoisomerase inhibitors, DNA crosslinking agents, Antimetabolites, and microtubule-targeting drugs;
Checkpoint inhibitors;
Cytotoxic antibiotics;
Alkylating agents;
immunomodulating agents;
Small molecule drugs; and
antibodies that stimulate the immune response to a given cancer.

The skilled person will know of other types of chemotherapeutics and also specific drugs. Thus, the above list is a non-limiting list of chemotherapeutics according to the present invention, which may cause release of cellular components, which may then function as STING stimulatory agents in vivo.

Similar, the skilled person will know of different types of radiation therapies and immunotherapies.

In yet another embodiment, the composition or combination, kit is administered intravenously (IV), intratumorally (IT), or subcutaneously (SC).

### In Vitro Use

The composition or combination or kit according to the invention may also find use in vitro. Thus, an aspect of the present invention relates to an in vitro method of increasing STING expression in a cell, said method comprising contacting said cell with a composition or combination or kit according to the present invention.

In another aspect, the invention relates to the use of a composition, combination, or kit according to the invention, for increasing STING expression in vitro in a cell.

### Additional Aspects

In an additional aspect, the invention relates to a method of preventing, treating and/or ameliorating a cancer in a subject in need thereof, said method comprising administering a composition or combination or kit according to the invention to a subject in need thereof, e.g. together with a STING stimulatory agent.

In yet an additional aspect, the invention relates to a method of preventing, treating and/or ameliorating a cancer in a subject in need thereof, wherein said subject has undergone cancer therapy or is undergoing cancer therapy, or who is scheduled for cancer therapy, with a different therapy, such as with chemotherapeutics, immunotherapy and/or by radiation, said method comprising administering a composition, combination, or kit according to the invention. Again, phrased in another way, the composition or combination according to the invention (without an exogenous STING stimulatory agent), may be for use in combinatorial cancer treatment or amelioration.

### Guide RNA

The present invention also discloses unique guide RNAs (such as sgRNA) which can be used for activating STING expression. Thus, an aspect of the invention relates to an isolated guide RNA comprising a sequence of at least 15 nucleotides, preferably 19-21 nucleotides such as 20 nucleotides, which is complementary to STING DNA within SEQ ID NO: 28, such as within position 200-900 of SEQ ID NO: 28, preferably within position 240-840, and more preferably within position 240-420 and even more preferably within position 260-380 and most preferably within position 270-300 of SEQ ID NO: 28. As outlined in the example section (e.g. example 4), the identified sgRNAs bind within this region.

The PAM, also known as the protospacer adjacent motif, is a short specific sequence following the target DNA sequence that is important for recognition and cleavage by a Cas nuclease.

The PAM is positioned about 2-6 nucleotides downstream of the DNA sequence targeted by the guide RNA and the wt Cas cuts 3-4 nucleotides upstream of it. In S. pyogenes, for example, Cas9 recognizes a 5′-NGG-3′ PAM (where “N” can be any nucleotide base).

Another critical function of PAM is that the Cas nuclease will search for it before unravelling the viral DNA in order to cut. When Cas identifies the correct PAM, it will then check to see if the upstream region matches the guide RNA before it makes the edit. Thus, also for dCAS the PAM region may be important for binding.

Thus, in an embodiment, the guide RNA binds upstream to a PAM sequence, such as “5′-NGG-3′”. It is to be understood that the guide RNA binds to the opposite strand of the indicated PAM sequence. In yet an embodiment, the 3′ end of the hybridizing part of the guide RNA binds 1-10 nucleotides upstream of the 5′-end of the PAM sequence, such as 1-6 nucleotides, preferably 2-6 nucleotides, more preferably 2-4 nucleotides upstream of the 5′-end of the PAM sequence.

In an embodiment, the isolated guide RNA comprises a sequence of at least 15 nucleotides, which is complementary to STING DNA within SEQ ID NO: 28 or the complementary sequence of SEQ ID NO: 28, wherein the guide RNA is complementary to STING DNA within SEQ ID NO: 28 or the complementary sequence of SEQ ID NO: 28, within 50 nucleotides upstream or downstream of the 5′-end or 3′-end of any of SEQ ID NO's: 1-5, preferably SEQ ID NO's: 4-5, more preferably SEQ ID NO: 4.

In an embodiment, the guide RNA is complementary to STING DNA within SEQ ID NO: 28 or the complementary sequence of SEQ ID NO: 28, within 40 nucleotides, such as within 30 nucleotides, preferably within 20 nucleotides and more preferably within 10 nucleotides upstream or downstream of the 5′-end or 3′-end of any of SEQ ID NO's: 1-5, preferably SEQ ID NO's: 4-5, more preferably SEQ ID NO: 4.

In yet another embodiment, the isolated guide RNA comprises
a) a sequence selected from the group consisting of SEQ ID Nos: 1-5;
b) a sequence complementary to a sequence selected from the group consisting of SEQ ID Nos: 1-5; or
c) a sequence according to a) or b) comprising 1-3 substitutions.

In a preferred embodiment the isolated guide RNA comprises a sequence identical or complementary to SEQ ID NO: 4, preferably identical to SEQ ID NO: 4.

Guide RNAs also comprises a (Cas9) nuclease-recruiting sequence (“tracrRNA”/scaffold sequence). Thus, in another embodiment, the guide RNA further comprises a Cas9 nuclease-recruiting sequence (“tracrRNA”/scaffold). SEQ ID NO: 32 is an example of a scaffold sequence and corresponds to the one used in the example section.

In a related aspect, the invention relates to the use of said guide RNA in CRISPR systems, preferably CRISPRa systems, such as for activating STING expression.

### Items of the Invention

1. A composition or combination comprising
a)
   an RNA molecule encoding an RNA-guided protein, such as a deactivated RNA-guided endonuclease, such as dCas, preferably dCas9; or
   an RNA-guided protein, such as a deactivated RNA-guided endonuclease, such as dCas, preferably dCas9; and
b) a guide RNA complementary to a part of STING DNA; and
c) a transcriptional activator, preferably
   encoded by the RNA molecule encoding the RNA-guided protein; or
   fused to the RNA-guided protein.
2. The composition or combination according to item 1, comprising an RNA molecule encoding an RNA-guided protein, such as encoding for dCas9.
3. The composition or combination according to item 1 or 2, being a CRISPR activation (CRISPRa) construct.
4. The composition or combination according to any of the preceding items, wherein the guide RNA binds to STING DNA within SEQ ID NO: 28 or the complementary sequence of SEQ ID NO: 28, such as within position 200-900 of SEQ ID NO: 28 or the complementary sequence of SEQ ID NO: 28, preferably within position 240-840, and more preferably within position 240-420 and even more preferably within position 260-380 and most preferably within position 270-300 of SEQ ID NO: 28 or the complementary sequence of SEQ ID NO: 28.
5. The composition or combination according to any of the preceding items, wherein the guide RNA comprises one or more oligonucleotide sequences selected from the group consisting of SEQ ID NO's: 1-5, more preferably SEQ ID NO: 4.
6. The composition or combination according to any of the preceding items, wherein the deactivated RNA-guided endonuclease is a dCas, such as a Cas9, such as SpCas9, SaCas9, or a Cas12, such as LbCas12a, AsCas12a, or Cas12f1, preferably dCas9 and more preferably Sp-dCas9.
7. The composition or combination according to any of the preceding items, wherein the 3′ end of the hybridizing part of the guide RNA binds 1-10 nucleotides upstream of the 5′-end of the PAM sequence, such as 1-6 nucleotides, preferably 2-6 nucleotides, more preferably 2-4 nucleotides upstream of the 5′-end of the PAM sequence.
8. The composition or combination according to any of the preceding items, wherein transcription activator is placed/fused in a spatial orientation, which allows it to affect the transcription of the STING gene.
9. A kit or combination comprising
a) a composition or combination according to any of items 1-8; and
b) a STING stimulatory agent.
10. The kit or combination according to item 9, wherein the STING stimulatory agent is selected from the group consisting of
nucleic acids, such as cyclic-di-nucleotide (CDNs), such as cyclic 2′3′ GMP-AMP (cGAMP), such as chemical stabilized forms of 2′3′ cGAMP;
enzymatic resistant double stranded DNA supporting intracellular cGAS activity;
DNA damage repair (DDR) inhibitors including but not limited to: PARP inhibitors; ATM inhibitors, Topoisomerase inhibitors, DNA crosslinking agents, microtubule-targeting drugs, or antimetabolites;
Small molecules capable of binding to and activating STING;
Checkpoint inhibitors; and/or
ENPP1 inhibitors.
11. The composition or combination according to any of items 1-8, or the kit or combination according to item 9 or 10 for use as a medicament.
12. The composition or combination according to any of items 1-8, or the kit or combination according to item 9 or 10 for use in the treatment or alleviation of cancer.
13. The composition or combination for use according to item 12, wherein the cancer is associated with no or low STING activity, such as insufficient STING activity, increased chromosomal instability and/or elevated cGAMP production.
14. An isolated guide RNA comprising a sequence of at least 15 nucleotides, preferably 19-21 nucleotides such as 20 nucleotides, which is complementary to STING DNA within SEQ ID NO: 28 or the complementary sequence of SEQ ID NO: 28, such as within position 200-900 of SEQ ID NO: 28 or the complementary sequence of SEQ ID NO: 28, preferably within position 240-840, and more preferably within position 240-420 and even more preferably within position 260-380 and most preferably within position 270-300 of SEQ ID NO: 28 or the complementary sequence of SEQ ID NO: 28.
15. The isolated guide RNA according to item 14, comprising a sequence which is identical to SEQ ID NO: 4.

It should be noted that embodiments and features described in the context of one of the aspects of the present invention also apply to the other aspects of the invention. For example, the modifications to guide RNAs described for one aspect also applies to other aspects relating to guide RNAs.

All patent and non-patent references cited in the present application, are hereby incorporated by reference in their entirety.

The invention will now be described in further details in the following non-limiting examples.

### Examples

### Example 1—Expression of Components in the cGAS-STING Signalling Pathway in Sarcoma Cell Lines

### Aim of Study

Assessment of basal expression of components in the cGAS-STING signalling pathway in human sarcoma cell lines.

### Materials and Methods

### Cell Culture

Human sarcoma cell lines HT-1080, SW872, SW982, and SK-LMS1 purchased from ATCC were all cultured at 37° C. with 5% CO₂ and under humidified conditions.

Cells we cultured in Dulbecco's modified eagle's medium (Sigma-Aldrich, Cat#: D6429) supplemented with 10% fetal bovine serum (Sigma-Aldrich, Cat#: F9665), 1% penicillin/streptomycin (Gibco, Cat#: 15140122), 1% L-glutamine (Thermo Fisher Scientific, Cat#: 25030024), and 1% non-essential amino acids solution (Gibco, Cat#: 11140035) (hereafter named DMEM complete). Cell lines were routinely tested for mycoplasma using the service of Eurofins Genomics.

### Real-Time PCR

RNA from cells was purified using RNeasy Mini kit (Qiagen, Cat#: 74104) according to manufacturer's guidelines. cDNA was synthesized using iScript cDNA Synthesis Kit (Bio-Rad, Cat#: 1708891) according to manufacturer's guidelines using 1 μg of RNA. The cDNA was diluted to a concentration of 5 ng/μL. Real-time PCR was carried out using 1 μL of 5 ng/μL cDNA in a total volume of 10 μL using TaqMan Fast Advanced master mix (Applied Biosystems, Cat#: 4444557) and TaqMan Gene expression assays (Applied Biosystems) for the following genes: MB21D1(cGAS) (hs00403553_m1), IFI16 (hs00194261_m1), TMEM173 (STING) (hs00736958_m1), TBK1 (hs00179410_m1), IRF3 (hs01547283_m1), DAG1 (hs00189308_m1). Samples were run in both biological and technical duplicates on a 384 well plate (Hounisen, Cat#: 72.1985.202). The analysis was performed using the LightCycler 480 software version LCS480 1.5.1.62(Roche).

### Western Blotting

Cells were lysed in RIPA buffer (Thermo Fisher Scientific, Cat#: 89901) supplemented with 10 mM NaF (VWR, Cat#: J60251.AE), pierce protease and phosphatase inhibitor (Thermo Fisher Scientific, Cat#: A32961), protease inhibitor cocktail (Roche, Cat#: 11873580001), and benzonase (Millipore, Cat#: E1014-25KU) in a volume of 1 μL/mL lysis buffer. Cell lysates were diluted 1:1 with Laemmli Lysis Buffer (Sigma-Aldrich, Cat#: 38733). The lysates were denatured at 95° C. for 5 minutes and 30 μL lysate were separated on a 10% Criterion Precast protein gel (Bio-Rad, Cat#: 5671034) with MOPS SDS running buffer (Invitrogen, Cat#: NP0001). The proteins we transferred to a 0.2 μm PVDF membrane (Bio-Rad, Cat#: 1704157) using a Trans-Blot Turbo Transfer System (Bio-Rad). A TBS wash buffer (fisher scientific, Cat#: BP2471-500) supplemented with 0.05% Tween-20 (Sigma-Aldrich, Cat#: P1379) (TBS-T) was used for washing the membranes. Membranes were blocked in 5% skim-milk (Sigma-Aldrich, Cat#: 70166) in TBS-T.

Membranes were incubated ON at 4° C. with the following primary antibodies diluted 1:1000 and 1:10000 (only vinculin) in 5% BSA (Roche, Cat#:

10735086001) in TBS-T: anti-cGAS (Cell Signaling Technologies, clone: D1D3G, Cat#: 15102S), anti-STING (Cell Signaling Technologies, clone: D2P2F, Cat#: 13647S), anti-IFI16 (Santa Cruz Biotechnology, clone: 1G7, Cat#: sc-8023), anti-TBK1 (Cell Signaling Technologies, Cat#: 3013S), anti-IRF3 (Cell Signaling Technologies, clone: D83B9, Cat#: 4302S), anti-vinculin (Sigma Life Sciences, Cat#: V9131). The following day, the membranes were incubated for 1 h at RT with the following secondary antibodies diluted 1:7500 in 5% skim milk in TBS-T: Peroxidase AffiniPure F(ab′)₂ Fragment Donkey Anti-Mouse IgG (Jackson Immuno Research, Cat#: 715-036-150), Peroxidase AffiniPure F(ab′)₂ Fragment Donkey Anti-Mouse IgG (Jackson Immuno Research, Cat#: 711-036-152). The membranes were developed using SuperSignal West Femto Maximum Sensitivity Substrate (Thermo Scientific, Cat#: 34095) or Clarity Western ECL Substrate (Bio-Rad, Cat#: 1705060).

### Results

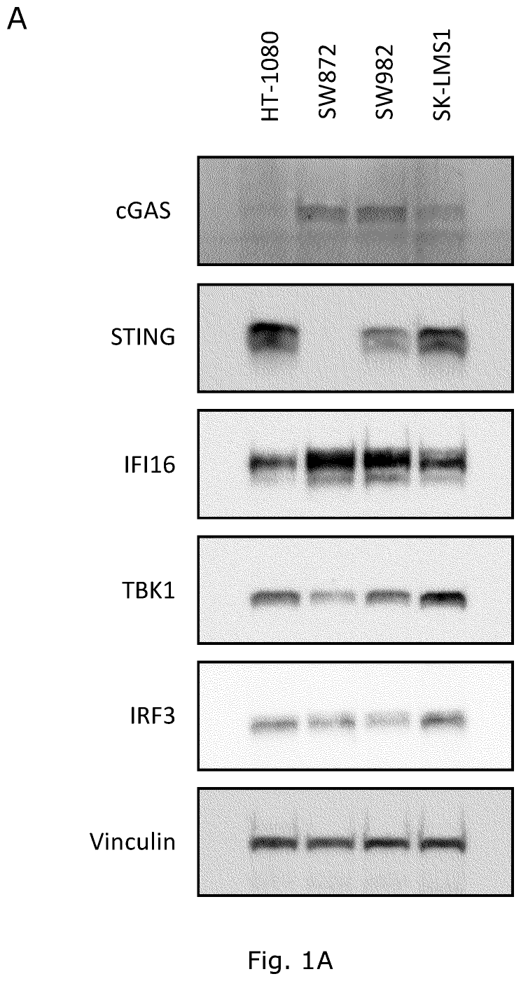

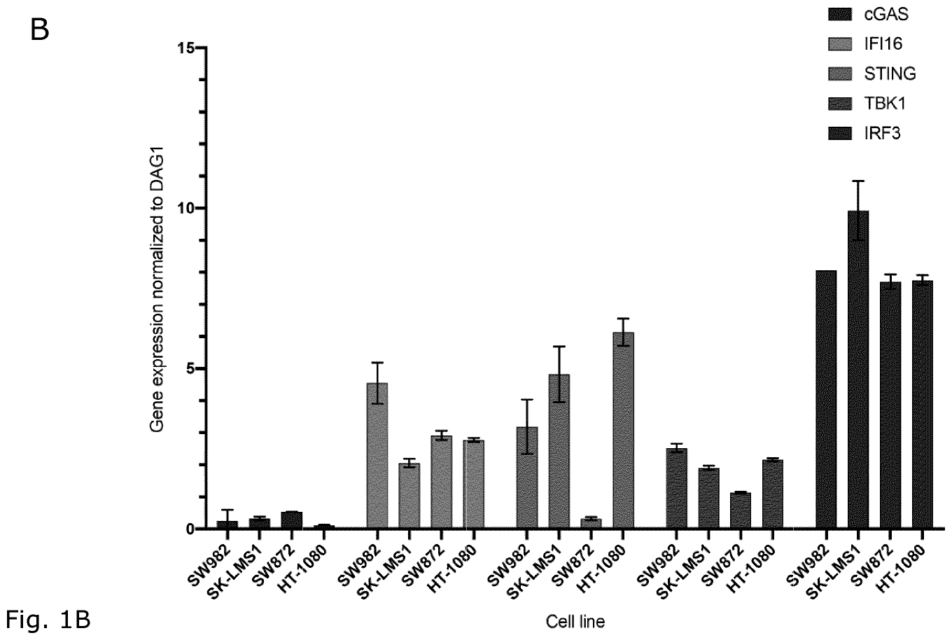

In order to assess endogenous protein expression of cGAS-STING signalling pathway components in human sarcoma cell lines, we made cellular protein and RNA lysate of each cell line. Protein lysates were loaded and run on an SDS-Page gel, followed by immunoblotting of cGAS, STING, IFI16, TBK1, IRF3, and vinculin as loading reference marker (FIG. 1A). The immunoblotting demonstrated divergent protein expression of both STING and cGAS in all four cancer cell lines. This image was confirmed by quantitative PCR, indicating that all cancer cell lines had very low cGAS expression and one cancer cell line, SW872, had epigenetic silenced STING expression (FIG. 1B).

### Conclusion

In summary, the data demonstrates that STING expression is suppressed in some cancer cell lines but not all.

### Example 2—Activating the cGAS-STING Signaling Pathway in Sarcoma Cancer Cell Lines

### Aim of Study

Assessment of the functional effect of activating the cGAS-STING signalling pathway in human sarcoma cancer cell lines.

### Materials and Methods

### Cell Culture

Human sarcoma cell lines HT-1080, SW872, SW982, and SK-LMS1 purchased from ATCC were all cultured at 37° C. with 5% CO₂ and under humidified conditions.

Cells we cultured in Dulbecco's modified eagle's medium (Sigma-Aldrich, Cat#: D6429) supplemented with 10% fetal bovine serum (Sigma-Aldrich, Cat#: F9665), 1% penicillin/streptomycin (Gibco, Cat#: 15140122), 1% L-glutamine

(Thermo Fisher Scientific, Cat#: 25030024), and 1% non-essential amino acids solution (Gibco, Cat#: 11140035) (hereafter named DMEM complete). Cell lines were routinely tested for mycoplasma using the service of Eurofins Genomics.

### In Vitro Stimulation of Cell Culture

For stimulation in vitro, 30,000 or 60,000 cells were seeded per well in a 24-well plate one day prior to stimulation. Cells were stimulated by transfection using Lipofectamine 2000 transfection reagent (Invitrogen, Cat#: 11668019) combined with the following agents: 2 μg/mL HT-DNA (Sigma-Aldrich, Cat#: D6898), 8 μg/mL 2′3′-cGAMP (Invivogen, Cat#: tlrl-nacga23-5), and 40 ng/ml Poly(I:C)-LMW (Invivogen, Cat#: tlrl-picw). An untreated control and a lipofectamine 2000 control were included. All samples were run in biological duplicates or triplicates. HT-DNA and 2′3′-cGAMP were mixed with lipofectamine 2000 in a ratio of 1:1 according to manufacturer's guidelines and Poly(I:C) was mixed with the same amount of lipofectamine as used for HT-DNA. For stimulation, the growth media was removed, and fresh growth media containing the stimulation and transfection agent was added and left at 37° C. with 5% CO₂ and under humidified conditions for 20 or 24 hours. Supernatants were harvested and used for cytokine detection assays.

### Mesoscale

Supernatants from cells stimulated with HT-DNA and Poly(I:C) were analyzed for Interferonβ (IFNβ), CXCL10 and IL-6 using Meso Scale Discovery multiplex Enzyme-linked Immunosorbent assay.

### Enzyme-Linked Immunosorbent Assay (ELISA)

Supernatants from cells stimulated with 2′3′-cGAMP were analyzed for CXCL10 and IL-6 using sandwich ELISA (RnD Systems, Cat#: DY299 and Cat#: DY206). CCL5 content in supernatants from cells stimulated with HT-DNA and Poly(I:C) were analyzed using sandwich ELISA (RnD Systems, Cat#: DY278). All were carried out according to manufacturer's guidelines.

### Functional Type I Interferon (IFN) Assay

Functional type I IFN in supernatants from cells stimulated with 2′3′-cGAMP was quantified using a type I interferon reporter cell assay (HEK-Blue IFN-α/β) (Invivogen, Cat#: hkb-ifnab) according to manufacturer's guidelines. HEK-Blue IFN-α/β cells were cultured in Dulbecco's modified eagle's medium (Sigma-Aldrich, Cat#: D6429) supplemented with 10% fetal bovine serum (Sigma-Aldrich, Cat#: F9665), 1% penicillin/streptomycin (Gibco, Cat#: 15140122), 1% L-glutamine (Thermo Fisher Scientific, Cat#: 25030024), 100 μg/mL normocin (Invivogen, Cat#: ant-nr-1), 30 μg/mL blasticidin (Invivogen, Cat#: ant-bl-1), and 100 μg/mL zeocin (Invivogen, Cat#: ant-zn-1). 30.000 cells were seeded per well in a 96-well plate in 150 μL media without blasticidin and zeocin. The following day, supernatants for analysis were added in a volume of 50 μL. HEK-Blue IFN-α/βcells express secreted alkaline phosphatase under control of the IFN-α/β inducible ISG54 promoter. SEAP activity was assessed the following day by mixing supernatants with Quanti-Blue (Invivogen, Cat#: rep-qb2) according to manufacturer's guidelines and measuring optical density at 620 nm on a microplate reader. The concentration of type I IFN was determined from a standard curve of IFN-a (PBL Assay Science, Cat#: 11100-1).

### Results

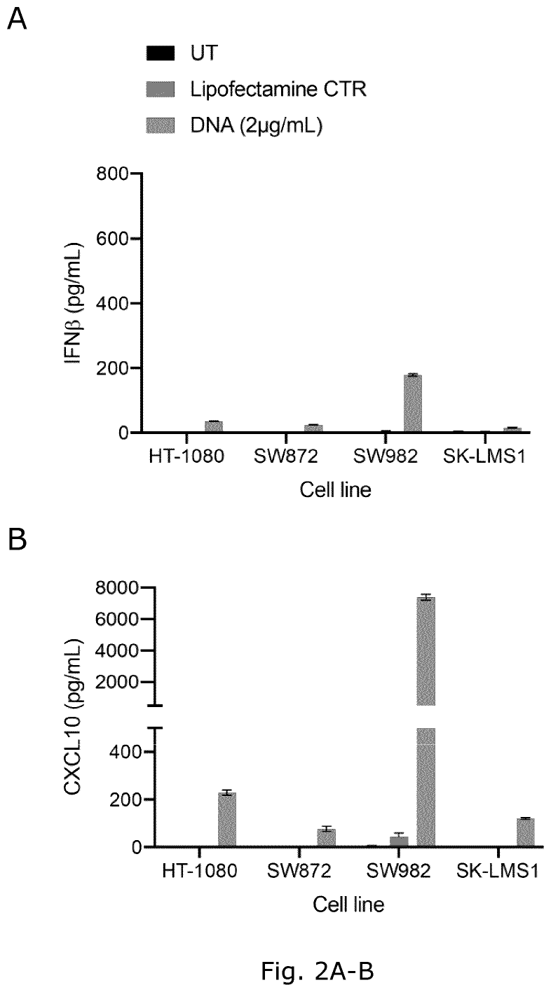

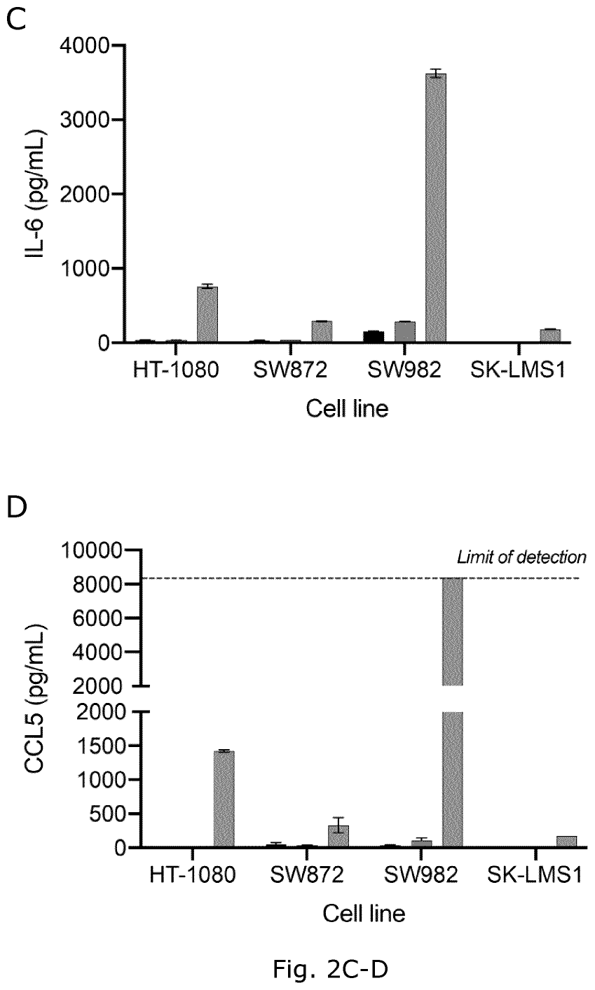

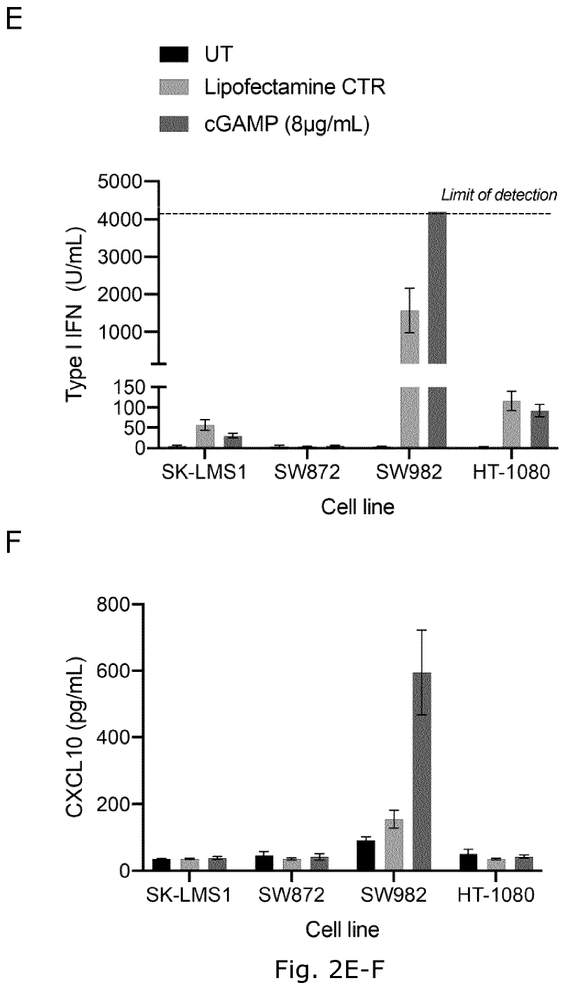

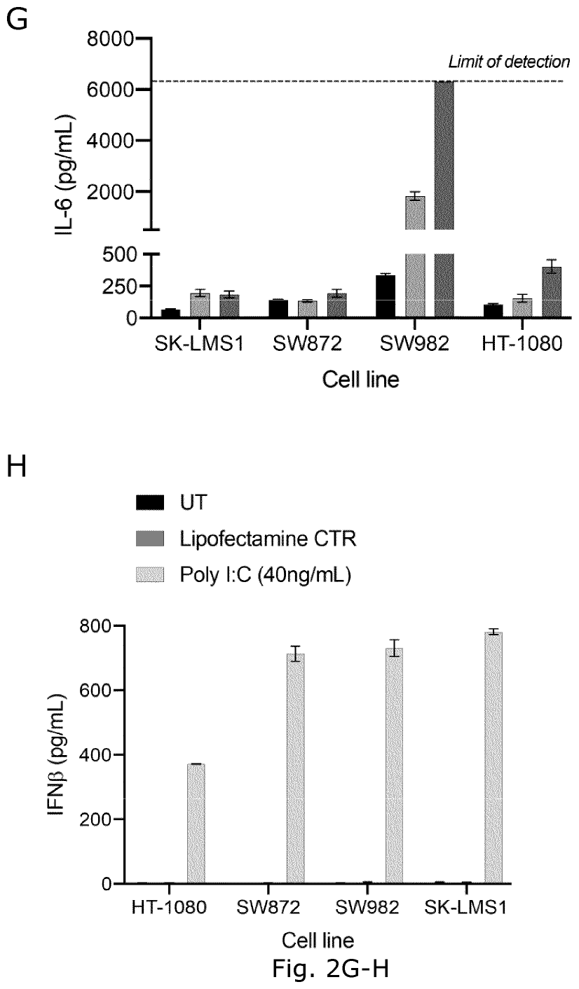

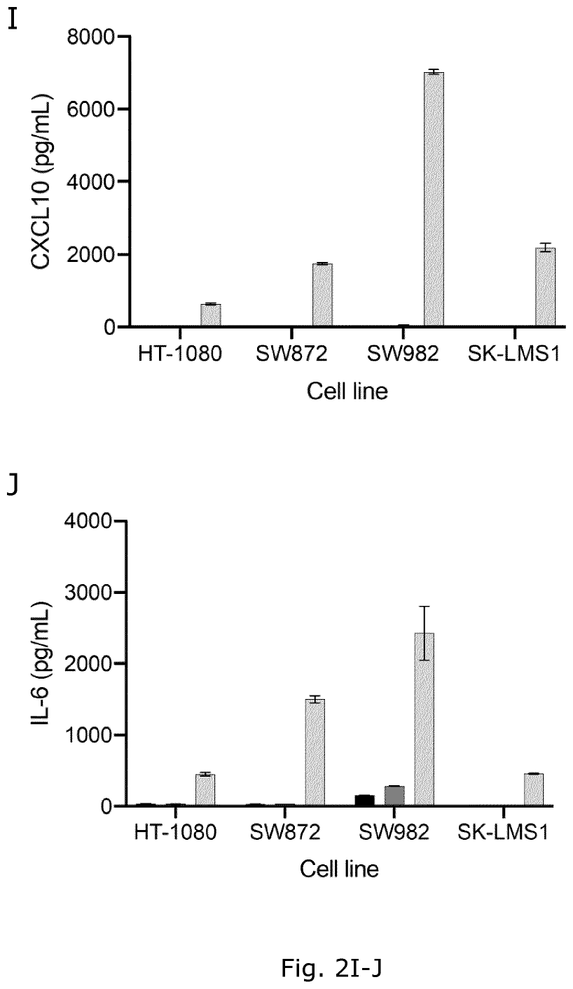

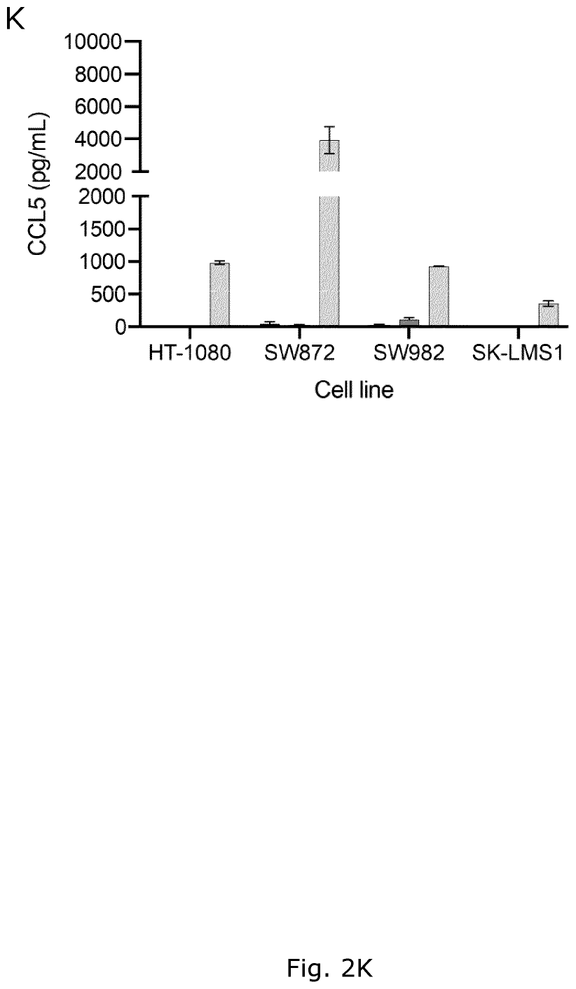

In order to evaluate endogenous cGAS-STING signalling pathway activity, we stimulated the four sarcoma cancer cells lines with a cGAS agonist in form of double-stranded DNA transfected by lipofectamine (FIG. 2A-D); STING agonist 2′3′ cGAMP (FIG. 2E-H) or a none-related innate immune agonist targeting the RIG-I pathway, Poly(I:C) (FIG. 2H-K).

After 20-24 hours of activation, cell responsiveness to agonists were determined by the secretion levels of IFNb, CXCL10, IL6 or CCL5. Importantly, SW872 was the only cell line not responding to neither cGAS nor STING agonist activation. As shown in example 1, SW872 had no obvious STING protein expression. However, all four cell lines responded to the RIG-I agonist Poly(I:C) confirming that the cell lines had the capacity to secrete the cytokines selected in the assay.

### Conclusion

In summary, these data demonstrate that suppression of STING expression occurs and limits the ability of the cancer cells to initiate an immunological response to agonists specifically targeting cGAS and STING pathway.

### Example 3—Theoretical Background of the Invention

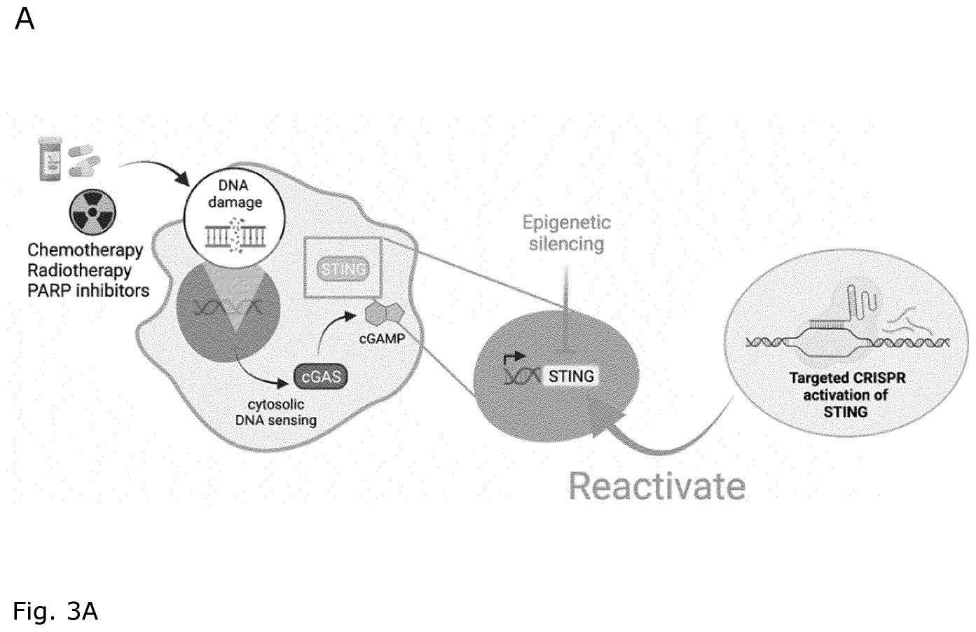

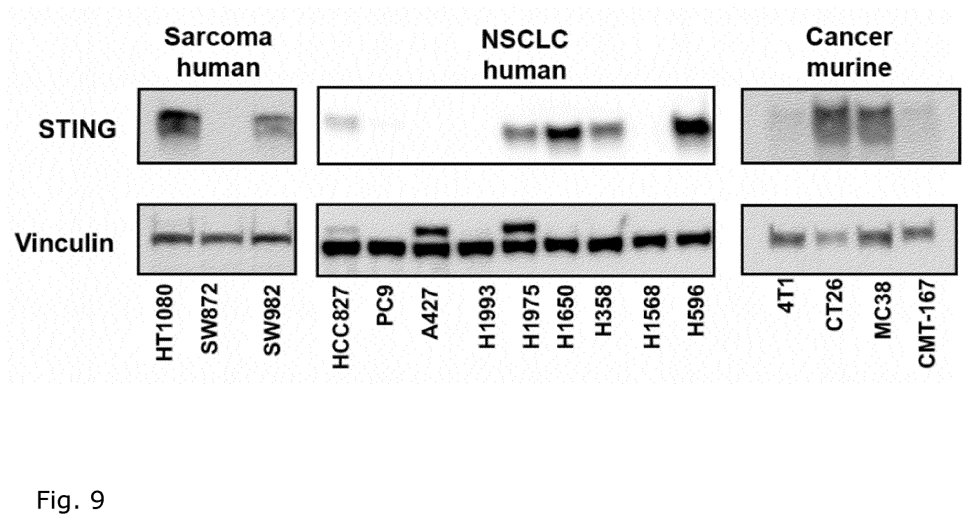

It is remarkable to observe that in many human and murine cancer cell lines a selective epigenetic silencing of STING expression occurs (FIGS. 1 and 9). This clearly suggests that cancer cells have developed immunological escape mechanisms that limit local immune responses and bypass cytotoxic T-cell surveillance by downregulating genes of interest. To increase the success rate of future immunotherapies, we hypothesize that it becomes important to counteract STING silencing, such as by using targeted CRISPR activation of STING (FIG. 3A).

With the discovery of the CRISPR-Cas9 technology, the field of genetic engineering and reprogramming has been revolutionized and it provides a means for precise genetic engineering in cells.

For the CRISPRa platform, a “dead Cas9” mutant (dCas) is utilized in which the nucleolytic activity is disabled, but the protein can still bind DNA. The dCas9 is fused to transcriptional activators, and a designed single-guide RNA molecule (sgRNA) allows for targeted localization of the dCas9-activator fusion to a specific gene. This complex will override gene silencing and allow for transient gene expression for days, depending on the target and cell type (FIG. 3B).

### Example 4—Reactivation of Genetically Silenced STING in the Sarcoma Cell Line SW872 Using CRISPRa Technology

### Aim of Study

Exploring the possibility to reactivate genetically silenced STING in the sarcoma cell line SW872 using CRISPRa technology.

### Materials and Methods

CRISPR activation of STING in vitro

To upregulate STING, CRISPR activation technique was used. Synthetic guideRNAs (sgRNAs) are used to guide a deactivated Cas9 (dCas9) to a site in the vicinity of the transcriptional start site of STING. Attached to the dCas9 are three transcription activators (VP64, Rta, p65; collectively termed VPR), which can activate transcription of the target gene. The dCas9-VPR is delivered as mRNA and is translated by the translation apparatus in the host cell.

To test the ability of the individual sgRNAs to upregulate STING expression, each sgRNA was tested individually as well as all 5 sgRNAs together in the human sarcoma cell line SW872 or HT1080. The sequence of the DNA binding part of the 5 sgRNAs were the following:

#1:
(SEQ ID NO: 1)
GGUUCCUACCUCCCUUCCUG,
#2:
(SEQ ID NO: 2)
UUUACUGGUGCUGGGAAGGA,
#3:
(SEQ ID NO: 3)
GAGUGUGUGGAGUCCUGCUC,
#4:
(SEQ ID NO: 4)
CAGAAACCGGCAGGCUCUCU,
#5:
(SEQ ID NO: 5)
GAGGAGGGGCACAGAGGAAU.

The scaffold part of the sgRNAs is listed in SEQ ID NO 32.

sgRNAs were purchased from Synthego and comprise 2′-O-Methyl at the three first and last bases as well as 3′ phosphorothioate bonds between the first three and last two bases.

Apart from the mRNA+sgRNA-electroporated cells, a mock control was included which went through the same treatment except no sgRNA and dCas9-VPR mRNA was added.

dCas9-VPR mRNA (SEQ ID NO: 29 as produced by in vitro transcription) (or purchased from Horizon Discovery, Cat#: CAS12211) comprises full substitution of uridine with pseudouridine and cotranscriptional capping with CleanCap AG in a 1:4 ratio between GTP and CleanCap.

For each sample 500,000 cells were resuspended in PBS, spun down at 300 xg for 5 minutes at room temperature (RT) and resuspended in 17 μL opti-MEM (Gibco, Cat#: 31985070). 1.9 μL of dCas9-VPR mRNA (Horizon Discovery, Cat#: CAS12211) (SEQ ID NO: 29) and 1 μg of sgRNA (1 μg of each sgRNA in the sample with all 5 sgRNAs) were added. The cell suspensions were added to an electroporation strip and electroporated on a Lonza 4D nucleofector using pulse code CM138. Afterwards, the electroporation strip containing the cells were left for 3 minutes on the bench top to allow cells to reform. The samples were diluted in preheated DMEM complete medium and cells were seeded at 50-100.000 cells per well in a 24-well plate and left for various days before harvest.

### Western Blotting

Cells were lysed in RIPA buffer (Thermo Fisher Scientific, Cat#: 89901) supplemented with 10 mM NaF (VWR, Cat#: J60251.AE), pierce protease and phosphatase inhibitor (Thermo Fisher Scientific, Cat#: A32961), protease inhibitor cocktail (Roche, Cat#: 11873580001), and benzonase (Millipore, Cat#: E1014-25KU) in a volume of 1 μL/mL lysis buffer. Cell lysates were diluted 1:1 with Laemmli Lysis Buffer (Sigma-Aldrich, Cat#: 38733). The lysates were denatured at 95° C. for 5 minutes and 15 μL lysate was separated on a 10% Criterion Precast protein gel (Bio-Rad, Cat#: 5671035) with MOPS SDS running buffer (Invitrogen, Cat#: NP0001). The proteins were transferred to a 0.2 μm PVDF membrane (Bio-Rad, Cat#: 1704157) using a Trans-Blot Turbo Transfer System (Bio-Rad). A TBS wash buffer (fisher scientific, Cat#: BP2471-500) supplemented with 0.05% Tween-20 (Sigma-Aldrich, Cat#: P1379) (TBS-T) was used for washing the membranes. Membranes were blocked in 5% skim-milk (Sigma-Aldrich, Cat#: 70166) in TBS-T.

Membranes were incubated ON at 4° C. with the following primary antibodies in 5% BSA (Roche, Cat#: 10735086001) in TBS-T: anti-STING (Cell Signaling Technologies, clone: D2P2F, Cat#: 13647S) diluted 1:1000 and anti-vinculin (Sigma Life Sciences, Cat#: V9131) diluted 1:10000. The following day, the membranes were incubated for 1 h at RT with the following secondary antibodies diluted 1:7500 in 5% skim milk in TBS-T: Peroxidase AffiniPure F(ab′)₂ Fragment Donkey Anti-Mouse IgG (Jackson Immuno Research, Cat#: 715-036-150), Peroxidase AffiniPure F(ab′)₂ Fragment Donkey Anti-Mouse IgG (Jackson Immuno Research, Cat#: 711-036-152). The membranes were developed using Clarity Western ECL Substrate (Bio-Rad, Cat#: 1705060).

### Real-time PCR

RNA from cells was purified using RNeasy Mini kit (Qiagen, Cat#: 74104) according to manufacturer's guidelines. cDNA was synthesized using iScript cDNA Synthesis Kit (Bio-Rad, Cat#: 1708891) according to manufacturer's guidelines using 100 ng of RNA. Real-time PCR was carried out using 1 μL of 5 ng/μL cDNA in a total volume of 10 μL using TaqMan Fast Advanced master mix (Applied Biosystems, Cat#: 4444557) and TaqMan Gene expression assays (Applied Biosystems) for the following genes: TMEM173 (STING) and EIF2B2. Samples were run in both biological and technical duplicates on a 384 well plate (Hounisen, Cat#: 72.1985.202). The analysis was performed using the LightCycler 480 software version LCS480 1.5.1.62(Roche).

### Results

A series of sgRNAs, targeting various regions of the transcriptional start site of the human STING gene, was designed (SEQ ID NO's: 1-5).

These sgRNAs were combined with dCas9-VPR mRNA (SEQ ID NO: 29) and electroporated into the cell line SW872. After 48 hours, cells were lysed and STING expression was determined by immunoblotting (FIG. 4A) or qPCR (FIG. 4B).

As shown in FIG. 4A-B, all sgRNAs were able to activate STING expression, albeit with different efficiency. sgRNA#4 gave the highest expression level.

### Conclusion

In summary, these data demonstrate that STING can be genetically re-programmed to enable transient expression using several individual sgRNAs combined with dCas9-VPR expression within cancer cells. The example also reveals that the design of a single sgRNA is superior to the effect of using many different sgRNA in combination.

### Example 5—Reactivating Genetically Silenced STING in Sarcoma Cell Line SW872 and HT-1080 Using the CRISPRa Technology

### Aim of Study

Explore the possibility to reactivate genetically silenced STING in sarcoma cell lines SW872 and HT-1080 using the CRISPRa technology

Materials and methods

See example 4.

### Results

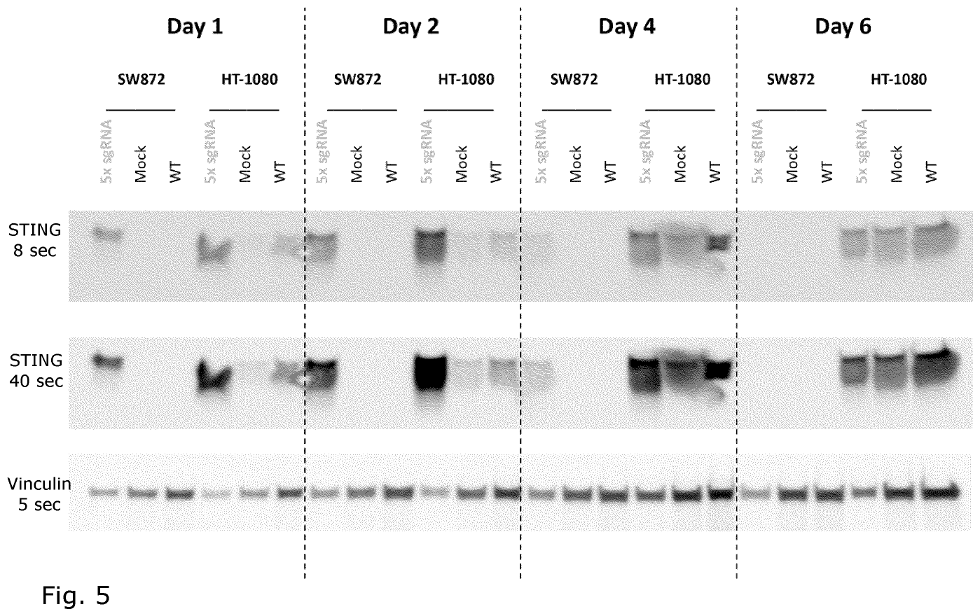

A series of proprietary sgRNAs targeting various regions surrounding the transcriptional start site of the human STING gene was designed (SEQ ID NO 1-5). These sgRNAs were combined with dCas9-VPR mRNA and electroporated into the cell line SW872 or HT-1080. As controls, cell samples were electroporated without the addition of sgRNA and dCas9-VPR (mock) as well as a cell sample left untreated (WT). After 24, 48, 96, and 144 hours, each cell population was lysed and STING expression was determined by immunoblotting for STING expression. The result demonstrated a rapid increase in STING protein expression in both cell lines after 24hrs and a peak in expression between 48 and 96 hours (FIG. 5). In HT-1080 which already did express some STING, we were able to induce this further showing very strong signals even at 96 hours post electroporation.

### Conclusion

In conclusion, these data demonstrate that STING expression can be genetically re-expressed using a combination of sgRNAs and dCas9-VPR both in cells with no endogenous STING expression and cells with a low STING expression.

### Example 6—Reactivating Genetically Silenced STING in Sarcoma Cell Line SW872 and HT-1080 Using the CRISPRa Technology

### Aim of Study

Explore the possibility to reactivate genetically silenced STING in sarcoma cell line SW872 and HT-1080 using the CRISPRa technology.

### Materials and Methods

See example 4.

### Results

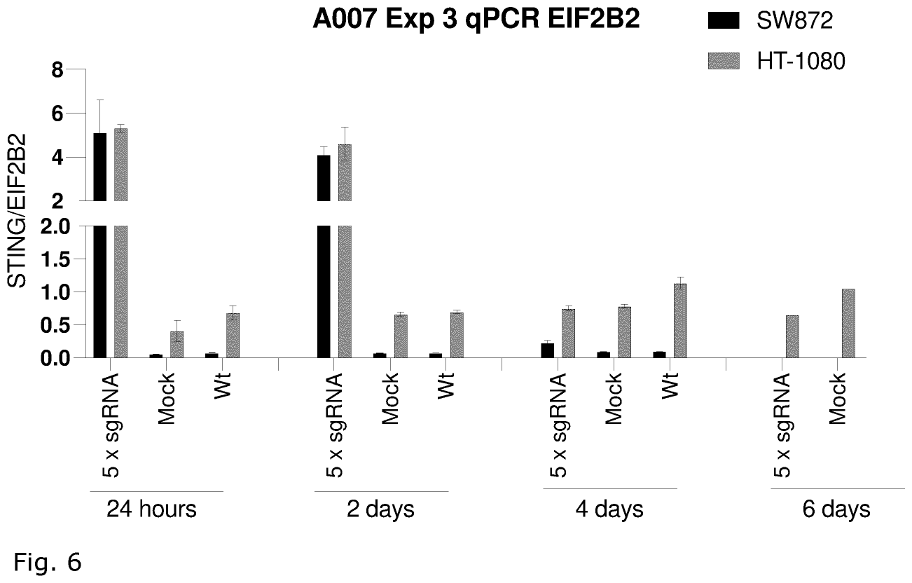

From the same experiment as demonstrated in example 5, we collected total RNA from each cell line population to determine the gene expression of STING in comparison to the reference gene (EIF2B2). The results demonstrated that STING mRNA expression was highly induced in both cell lines at 24 hours. After 96 hours, the expression of STING had disappeared in SW872, and reached its basal level in HT-1080 (FIG. 6).

### Conclusion

In conclusion, these data demonstrate that STING expression can be genetically re-expressed using a combination of sgRNAs and dCas9-VPR both in cells with no endogenous STING expression and cells with a low STING expression.

### Example 7—Immune Activation Potentials in Sarcoma Cell Line SW872 After STING-Targeting CRISPRa Reactivation

Aim of study

Explore the immune activation potential in sarcoma cell line SW872 after STING-targeted CRISPRa reactivation.

### Materials and Methods

### Cell Culture

Human sarcoma cell line SW872 was cultured in Dulbecco's modified eagle's medium (Sigma-Aldrich, Cat#: D6429) supplemented with 10% fetal bovine serum (Sigma-Aldrich, Cat#: F9665), 1% penicillin/streptomycin (Gibco, Cat#: 15140122), 1% L-glutamine (Thermo Fisher Scientific, Cat#: 25030024), and 1% non-essential amino acids solution (Gibco, Cat#: 11140035) (hereafter named DMEM complete). Cell lines were routinely tested for mycoplasma using the service of Eurofins Genomics.

### CRISPR Activation of STING In Vitro

The sequence of the DNA binding part of the sgRNA was

#4:
(SEQ ID NO: 4)
CAGAAACCGGCAGGCUCUCU.

sgRNAs were purchased from Synthego (see also example 4 for details).

Apart from the mRNA+sgRNA-electroporated cells, a mock control was included which went through the same treatment except no sgRNA and dCas9-VPR mRNA was added and a wild type (WT) control.

For each sample 500,000 cells were resuspended in PBS, spun down at 300 xG for 5 minutes at room temperature (RT) and resuspended in 17 μL opti-MEM (Gibco, Cat#: 31985070). 1.9 μL of dCas9-VPR mRNA (Horizon Discovery, Cat#: CAS12211) and 1 μg of sgRNA was added. The cell suspension was added to an electroporation strip and electroporated on a Lonza 4D nucleofector using pulse code CM138. Afterwards, the electroporation strip containing the cells was left for 3 minutes on the bench top to allow cells to reform. The samples were diluted in preheated DMEM complete and 30,000 wt cells and 60,000 mock and STING+ cells were seeded in a 24-well plate one day prior to stimulation to allow cells to adhere.

### In Vitro Stimulation of Cell Culture

Cells were stimulated by transfection using Lipofectamine 2000 transfection reagent (Invitrogen, Cat#: 11668019). The following agents were used for transfection: 1 μg/mL HT-DNA (Sigma-Aldrich, Cat#: D6898) and 4 μg/mL 2′3′-cGAMP (Invivogen, Cat#: tlrl-nacga23-5). An untreated control and a lipofectamine 2000 control were also included. All samples were run in biological triplicates. HT-DNA and 2′3′-cGAMP were mixed with lipofectamine 2000 in a ratio of 1:1 according to manufacturer's guidelines. For stimulation, the growth media was removed, and fresh growth media containing the stimulation and transfection agent was added and left at 37° C. with 5% CO₂ and under humidified conditions for 24 hrs. Supernatants were harvested and used for cytokine detection assays.

### Enzyme-Linked Immunosorbent Assay (ELISA)

Supernatants were analyzed for IL-6 using sandwich ELISA (RnD Systems, Cat#: DY206). CCL5 content in supernatants were analyzed using sandwich ELISA (RnD Systems, Cat#: DY278). All were carried out according to manufacturer's guidelines

### Functional Type I Interferon (IFN) Assay

Functional type I IFN in supernatants was quantified using a type I interferon reporter cell assay (HEK-Blue IFN-α/β) (Invivogen, Cat#: hkb-ifnab) according to manufacturer's guidelines.

HEK-Blue IFN-α/β cells were cultured in Dulbecco's modified eagle's medium (Sigma-Aldrich, Cat#: D6429) supplemented with 10% fetal bovine serum (Sigma-Aldrich, Cat#: F9665), 1% penicillin/streptomycin (Gibco, Cat#: 15140122), 1% L-glutamine (Thermo Fisher Scientific, Cat#: 25030024), 100 μg/mL normocin (Invivogen, Cat#: ant-nr-1), 30 μg/mL blasticidin (Invivogen, Cat#: ant-bl-1), and 100 μg/mL zeocin (Invivogen, Cat#: ant-zn-1). 30.000 cells were seeded per well in a 96-well plate in 150 μL media without blasticidin and zeocin. The following day, supernatants for analysis were added in a volume of 50 μL. HEK-Blue IFN-α/β cells express secreted alkaline phosphatase under control of the IFN-α/β inducible ISG54 promoter. SEAP activity was assessed the following day by mixing supernatants with Quanti-Blue (Invivogen, Cat#: rep-qb2) according to manufacturer's guidelines and measuring optical density at 620 nm on a microplate reader. The concentration of type I IFN was determined from a standard curve of IFN-α (PBL Assay Science, Cat#: 11100-1).

### Results

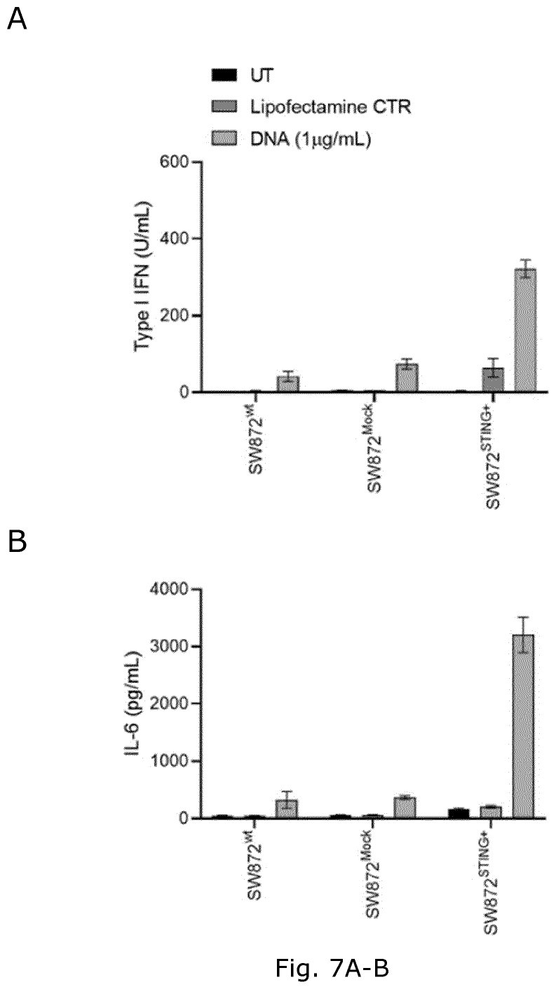

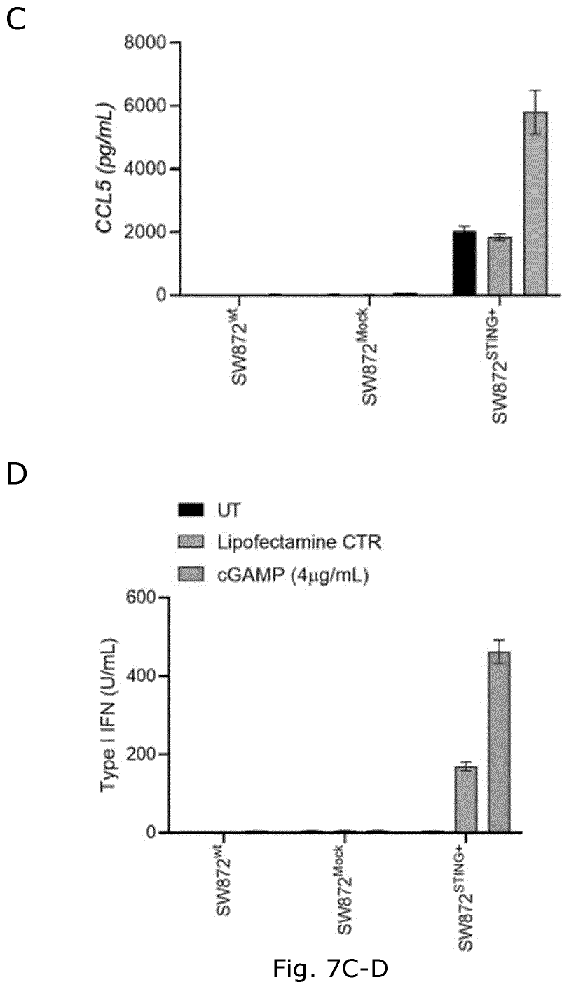

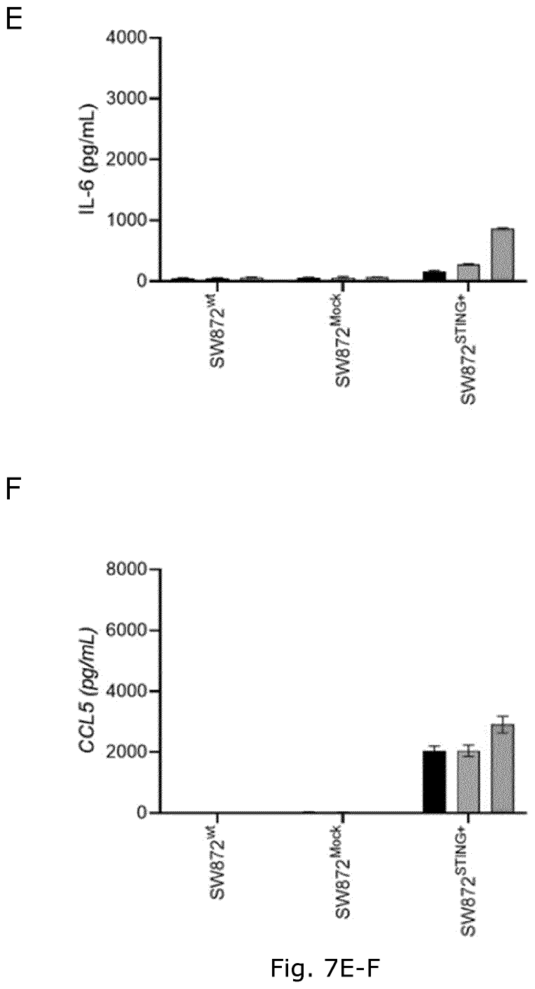

From the initial screenings, we identified sgRNA #4 (SEQ ID NO 4) to be equally or better than the combination of all five sgRNAs targeting STING. Therefore, we repeated an electroporation of SW872 with dCas9-VPR mRNA and either nothing (mock) or sgRNA #4 and after additional 24 hrs each cell conditions were stimulated with either HT-DNA (FIG. 7A-C) or 2′3′-cGAMP (FIG. 7D-F). The level of STING activation was determined after 24 hrs using a human type I IFN bioassay (FIG. 7A+D) or ELISA for IL6 (FIG. 7B+E) or CCL5 (FIG. 7C+F).

The results demonstrate that wildtype (wt) and mock treated SW872 cells were unresponsive to STING agonist stimulation, whereas CRISPRa reactivation of STING in SW872 made them secrete high cytokine levels of type I IFN, IL6, and CCL5.

### Conclusion

In summary, these data demonstrate that STING re-expression using a combination of targeted sgRNA with dCas9-VPR mRNA delivery can sensitize cancer cells to respond to STING activation and become immunologically active.

### Example 8—Immune Activation Potential in Sarcoma Cell Line SW872 After STING-Targeted CRISPRa Reactivation

### Aim of Study

Explore the immune activation potential in sarcoma cell line SW872 after STING-targeted CRISPRa reactivation.

### Materials and Methods

### Cell Culture

Human sarcoma cell line SW872 purchased from ATCC was cultured at 37° C. with 5% CO₂ and under humidified conditions.

Cells were cultured in Dulbecco's modified eagle's medium (Sigma-Aldrich, Cat#: D6429) supplemented with 10% fetal bovine serum (Sigma-Aldrich, Cat#: F9665), 1% penicillin/streptomycin (Gibco, Cat#: 15140122), 1% L-glutamine

(Thermo Fisher Scientific, Cat#: 25030024), and 1% non-essential amino acids solution (Gibco, Cat#: 11140035) (hereafter named DMEM complete). Cell lines were routinely tested for mycoplasma using the service of Eurofins Genomics.

### CRISPR Activation of STING in Vitro

To upregulate STING, CRISPR activation technique was used. Synthetic guideRNAs (sgRNAs) are used to guide a deactivated Cas9 (dCas9) to the vicinity of the transcriptional start site of the STING gene. Attached to the dCas9 are three transcription activators (VP64, Rta, p65), which can activate transcription of the target gene. The dCas9-VPR is delivered as mRNA and is translated by the translation apparatus in the host cell.

The DNA binding part of the sequence of the sgRNA was

#4:
(SEQ ID NO: 4)
CAGAAACCGGCAGGCUCUCU.

sgRNAs were purchased from Synthego (see also example 4 for details).

Apart from the mRNA+sgRNA-electroporated cells, a mock control was included which went through the same treatment except no sgRNA and dCas9-VPR mRNA was added and a wild type (WT) control.

For each sample 500,000 cells were resuspended in PBS, spun down at 300 xG for 5 minutes at room temperature (RT) and resuspended in 17 μL opti-MEM (Gibco, Cat#: 31985070). 1.9 μL of dCas9-VPR mRNA (Horizon Discovery, Cat#: CAS12211) and 1 μg of sgRNA was added. The cell suspension was added to an electroporation strip and electroporated on a Lonza 4D nucleofector using pulse code CM138. Afterwards, the electroporation strip containing the cells was left for 3 minutes on the bench top to allow cells to reform. The samples were diluted in preheated DMEM complete and 60,000 wt cells and 120,000 mock and STING+ cells were seeded in a 12-well plate one day prior to doxorubicin treatment to allow cells to adhere.

Doxorubicin treatment in vitro

Prior to analysing the immunological reponse to doxorubicin treatment (Sigma-Aldrich, Cat#: D1515-10 MG), and IC₅₀ viability concentration was determined for both wt cells and electroporated cells (mock and STING+). A two-fold dilution of doxorubicin including 10 different dilutions ranging from 16 μM to 0.03125 μM was added to a cell culture and viability was determined using CellTiter 96 AQueous One Solution Cell Proliferation Assay (Promega, Cat#: G3580) after 48 hrs of treatment.

Wt, mock and STING+ cells were treated with IC₅₀ values of doxorubicin in biological triplicates (IC₅₀ for wt: 0.5 μM and IC₅₀ for mock and STING+: 0.3 μM) and left for 48 hrs before harvest.

### Real-Time PCR

RNA from cells was purified using NucleoSpin RNA for the isolation of total RNA (Macherey-Nagel, Cat#: 740984.250M) according to manufacturer's guidelines. cDNA was synthesized using iScript cDNA Synthesis Kit (Bio-Rad, Cat#: 1708891) according to manufacturer's guidelines using 400ng of RNA. Real-time PCR was carried out using 1 μL of 10 ng/μL cDNA in a total volume of 10 μL using KAPA SYBR FAST (Roche, Cat#: KK4611) and the following targets and primers:

| | |
|---|---|
| Target | Primer SEQ ID NO |

| | | |
|---|---|---|
| TMEM173 | Forward | SEQ ID NO: 9 |
| (STING) | Reverse | SEQ ID NO: 10 |
| IL-6 | Forward | SEQ ID NO: 11 |
| | Reverse | SEQ ID NO: 12 |
| CXCL10 | Forward | SEQ ID NO: 13 |
| | Reverse | SEQ ID NO: 14 |
| CCL5 | Forward | SEQ ID NO: 15 |
| | Reverse | SEQ ID NO: 16 |
| IFNB1 | Forward | SEQ ID NO: 17 |
| | Reverse | SEQ ID NO: 18 |
| IFNL1 | Forward | SEQ ID NO: 19 |
| | Reverse | SEQ ID NO: 20 |
| EIF2B2 | Forward | SEQ ID NO: 21 |
| | Reverse | SEQ ID NO: 22 |

Samples were run in biological triplicates and technical duplicates on a 384 well plate (Hounisen, Cat#: 72.1985.202). The analysis was performed using the LightCycler 480 software version LCS480 1.5.1.62(Roche).

### Results

The cancer cell line SW872 was electroporated with dCas9-VPR mRNA and either nothing (mock) or sgRNA #4 and after additional 24 hrs each cell condition was exposed to the anthracycline chemotherapy drug doxorubicin. Doxorubicin's mode-of-action is to increase DNA damage leakage and this has shown to induce a STING-dependent immune response in conjunction to the cellular toxicity is has on cancer cells.

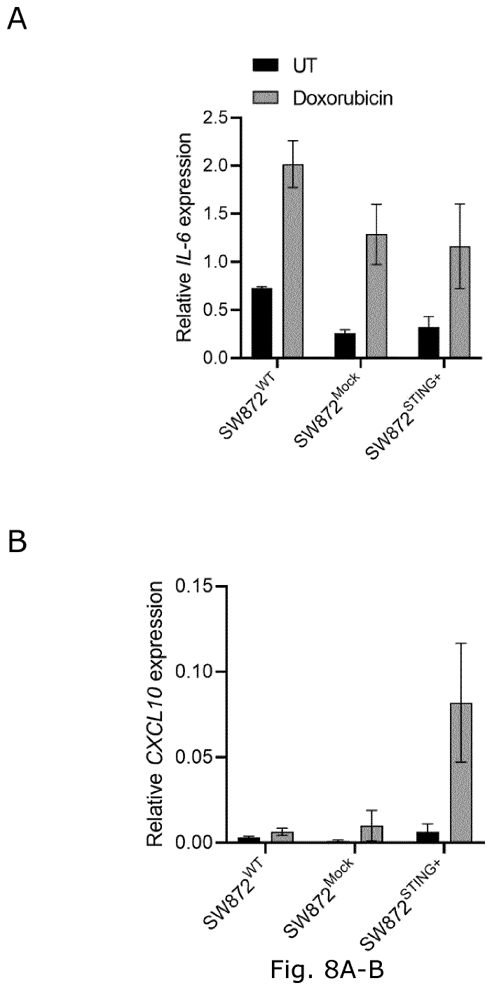

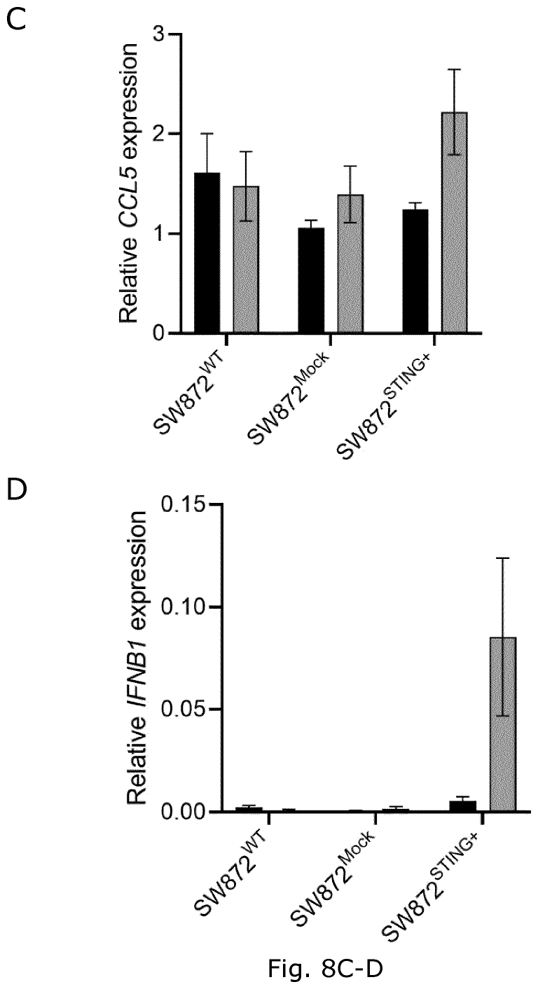

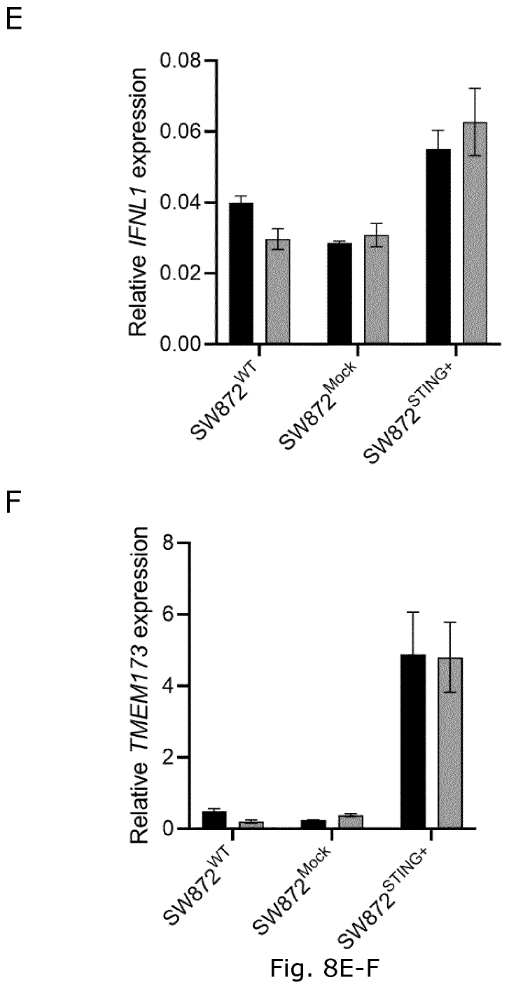

Supernatants were collected 48 hrs after doxorubicin treatment and immune activation was assessed by qPCR (FIG. 8A-E). The results demonstrate that CRISPRa targeting STING gene expression in SW872 cells (FIG. 8F) combined with doxorubicin treatment supported an increased gene expression of CXCL10, IFNb and CCL5 (FIG. 8B-D).

### Conclusion

In sum, these data demonstrate that STING re-expression using a combination of targeted sgRNA with dCas9-VPR mRNA delivery can sensitize cancer cells to respond to STING activation and become immunological active following treatment with chemotherapy drugs.

### Example 9—STING Expression in Human and Murine Cancer Cell Lines

### Aim of Study

Explore basal STING expression in human and murine cancer cell lines.

### Materials and Methods

### Cell Culture

Human cell lines HT-1080, SW872, SW982, HCC827, PC9, A427, H1993, H1975, H1650, H358, H1568, H596 and murine cell lines 4T1, CT26, MC38, CMT-167 were purchased from ATCC were all cultured at 37° C. with 5% CO₂ and under humidified conditions.

HT-1080, SW872, and SW982 cells we cultured in Dulbecco's modified eagle's medium (Sigma-Aldrich, Cat#: D6429) supplemented with 10% fetal bovine serum (Sigma-Aldrich, Cat#: F9665), 1% penicillin/streptomycin (Gibco, Cat#: 15140122), 1% L-glutamine (Thermo Fisher Scientific, Cat#: 25030024), and 1% non-essential amino acids solution (Gibco, Cat#: 11140035)

4T1, CT26, MC38, HCC827, PC9, H1993, H1975, H1650, H358, H1568, and H596 cells were cultured in Dulbecco's modified eagle's medium (Sigma-Aldrich, Cat#: D6429) supplemented with 10% fetal bovine serum (Sigma-Aldrich, Cat#: F9665), 1% penicillin/streptomycin (Gibco, Cat#: 15140122), 1% L-glutamine (Thermo Fisher Scientific, Cat#: 25030024),

A427, and CMT-167 were cultured in RPMI-1640 (Sigma-Aldrich, Cat#: R8758) supplemented with 10% fetal bovine serum (Sigma-Aldrich, Cat#: F9665), 1% penicillin/streptomycin (Gibco, Cat#: 15140122), and 1% L-glutamine (Thermo Fisher Scientific, Cat#: 25030024),

Cell lines were routinely tested for mycoplasma using the service of Eurofins Genomics.

### Western Blotting

Cells were lysed in RIPA buffer (Thermo Fisher Scientific, Cat#: 89901) supplemented with 10 mM NaF (VWR, Cat#: J60251.AE), pierce protease and phosphatase inhibitor (Thermo Fisher Scientific, Cat#: A32961), protease inhibitor cocktail (Roche, Cat#: 11873580001), and benzonase (Millipore, Cat#: E1014-25KU) in a volume of 1 μL/mL lysis buffer. Cell lysates were diluted 1:1 with Laemmli Lysis Buffer (Sigma-Aldrich, Cat#: 38733). The lysates were denatured at 95° C. for 5 minutes and 15 μL lysate were separated on a 10% Criterion Precast protein gel (Bio-Rad, Cat#: 5671034) with MOPS SDS running buffer (Invitrogen, Cat#: NP0001). The proteins we transferred to a 0.2 μm PVDF membrane (Bio-Rad, Cat#: 1704157) using a Trans-Blot Turbo Transfer System (Bio-Rad). A TBS wash buffer (fisher scientific, Cat#: BP2471-500) supplemented with 0.05% Tween-20 (Sigma-Aldrich, Cat#: P1379) (TBS-T) was used for washing the membranes. Membranes were blocked in 5% skim-milk (Sigma-Aldrich, Cat#: 70166) in TBS-T.

Membranes were incubated ON at 4° C. with the following primary antibodies diluted 1:1000 and 1:10000 (only vinculin) in 5% BSA (Roche, Cat#: 10735086001) in TBS-T: anti-STING (human) (Cell Signaling Technologies, clone: D2P2F, Cat#: 13647S), anti-STING (rodent) (Cell Signaling Technologies, clone: D1V5L, Cat#: 50494S) anti-vinculin (Sigma Life Sciences, Cat#: V9131). The following day, the membranes were incubated for 1 h at RT with the following secondary antibodies diluted 1:7500 in 5% skim milk in TBS-T: Peroxidase AffiniPure F(ab′)₂ Fragment Donkey Anti-Mouse IgG (Jackson Immuno Research, Cat#: 715-036-150), Peroxidase AffiniPure F(ab′)₂ Fragment Donkey Anti-Mouse IgG (Jackson Immuno Research, Cat#: 711-036-152). The membranes were developed using SuperSignal West Femto Maximum Sensitivity Substrate (Thermo Scientific, Cat#: 34095) or Clarity Western ECL Substrate (Bio-Rad, Cat#: 1705060).

### Results

Different human sarcoma and non-small cell lung cancer cell lines, as well as murine cancer cell lines were assessed by immunoblotting for basal STING expression (FIG. 9).

### Conclusion

In summary, these data demonstrate that STING is not constitutively expressed in cancer cells but approximately 50% of the tested cell lines lacked expression.

### Example 10—Reactivating Genetically Silenced STING in the Murine Lung Cancer Cell Line CMT-167 Using CRISPRa Technology

### Aim of Study

Exploring the possibility to reactivate epigenetically silenced STING in the murine lung cancer cell line CMT-167 using CRISPRa technology.

### Materials and Methods

### CRISPR Activation of STING In Vitro

Murine cell line CMT-167 were purchased from ATCC were all cultured at 37° C. with 5% CO₂ and under humidified conditions. CMT-167 were cultured in RPMI-1640 (Sigma-Aldrich, Cat#: R8758) supplemented with 10% fetal bovine serum (Sigma-Aldrich, Cat#: F9665), 1% penicillin/streptomycin (Gibco, Cat#: 15140122), and 1% L-glutamine (Thermo Fisher Scientific, Cat#: 25030024). Cell lines were routinely tested for mycoplasma using the service of Eurofins Genomics.

To test the ability of the individual sgRNA to upregulate STING expression, each sgRNA was tested individually as well as all 3 sgRNAs together. The sequence of the DNA binding part of the 3 sgRNAs were the following:

#6:
(SEQ ID NO: 6)
CCAGUCUCAGGAUGGUUGAG,
#7:
(SEQ ID NO: 7)
AGGUACUAGAAUUAAAUGAA,
#8:
(SEQ ID NO: 8)
GAAACAGGAUUAGAAGCCUU.

sgRNAs were purchased from Synthego (see also example 4 for details).

Apart from the mRNA+sgRNA-electroporated cells, a wt control and a mock control was included which went through the same treatment except no sgRNA and dCas9-VPR mRNA was added.

For each sample 500,000 cells were resuspended in PBS, spun down at 300 xg for 5 minutes at room temperature (RT) and resuspended in 17 μL opti-MEM (Gibco, Cat#: 31985070). 1.9 μL of dCas9-VPR mRNA (Horizon Discovery, Cat#: CAS12211) and 1 μg of sgRNA (1 μg of each sgRNA in the sample with all 3 sgRNAs) were added. The cell suspensions were added to an electroporation strip and electroporated on a Lonza 4D nucleofector using pulse code CM138. Afterwards, the electroporation strip containing the cells were left for 3 minutes on the bench top to allow cells to reform. The samples were diluted in preheated culture media and 50,000 cells were seeded per well in a 24-well plate and left for 48 hrs before harvest.

### Western Blotting

Cells were lysed in RIPA buffer (Thermo Fisher Scientific, Cat#: 89901) supplemented with 10 mM NaF (VWR, Cat#: J60251.AE), pierce protease and phosphatase inhibitor (Thermo Fisher Scientific, Cat#: A32961), protease inhibitor cocktail (Roche, Cat#: 11873580001), and benzonase (Millipore, Cat#: E1014-25KU) in a volume of 1 μL/mL lysis buffer. Cell lysates were diluted 1:1 with Laemmli Lysis Buffer (Sigma-Aldrich, Cat#: 38733). The lysates were denatured at 95° C. for 5 minutes and 15 μL lysate were separated on a 10% Criterion Precast protein gel (Bio-Rad, Cat#: 5671035) with MOPS SDS running buffer (Invitrogen,

Cat#: NP0001). The proteins we transferred to a 0.2 μm PVDF membrane (Bio-Rad, Cat#: 1704157) using a Trans-Blot Turbo Transfer System (Bio-Rad). A TBS wash buffer (fisher scientific, Cat#: BP2471-500) supplemented with 0.05% Tween-20 (Sigma-Aldrich, Cat#: P1379) (TBS-T) was used for washing the membranes. Membranes were blocked in 5% skim-milk (Sigma-Aldrich, Cat#: 70166) in TBS-T.

Membranes were incubated ON at 4° C. with the following primary antibodies in 5% BSA (Roche, Cat#: 10735086001) in TBS-T: anti-STING (rodent) (Cell Signaling Technologies, clone: D1V5L, Cat#: 50494S) diluted 1:1000 and anti-vinculin (Sigma Life Sciences, Cat#: V9131) diluted 1:10000. The following day, the membranes were incubated for 1 h at RT with the following secondary antibodies diluted 1:7500 in 5% skim milk in TBS-T: Peroxidase AffiniPure F(ab′)₂ Fragment Donkey Anti-Mouse IgG (Jackson Immuno Research, Cat#: 715-036-150), Peroxidase AffiniPure F(ab′)₂ Fragment Donkey Anti-Mouse IgG (Jackson Immuno Research, Cat#: 711-036-152). The membranes were developed using Clarity Western ECL Substrate (Bio-Rad, Cat#: 1705060).

### Real-Time PCR

RNA from cells was purified using NucleoSpin RNA for the isolation of total RNA (Macherey-Nagel, Cat#: 740984.250M) according to manufacturer's guidelines.

cDNA was synthesized using iScript cDNA Synthesis Kit (Bio-Rad, Cat#: 1708891) according to manufacturer's guidelines using 800 ng of RNA. Real-time PCR was carried out using 1 μL of 10 ng/μL cDNA in a total volume of 10 μL KAPA SYBR FAST (Roche, Cat#: KK4611) and the following targets and primers:

| | |
|---|---|
| Target | Primer SEQ ID NO |

| | | |
|---|---|---|
| TMEM173 | Forward | SEQ ID NO: 23 |
| (STING) | Reverse | SEQ ID NO: 24 |
| ppia | Forward | SEQ ID NO: 25 |
| | Reverse | SEQ ID NO: 26 |

Samples were run in both biological and technical duplicates on a 384 well plate (Hounisen, Cat#: 72.1985.202). The analysis was performed using the LightCycler 480 software version LCS480 1.5.1.62(Roche).

### Results

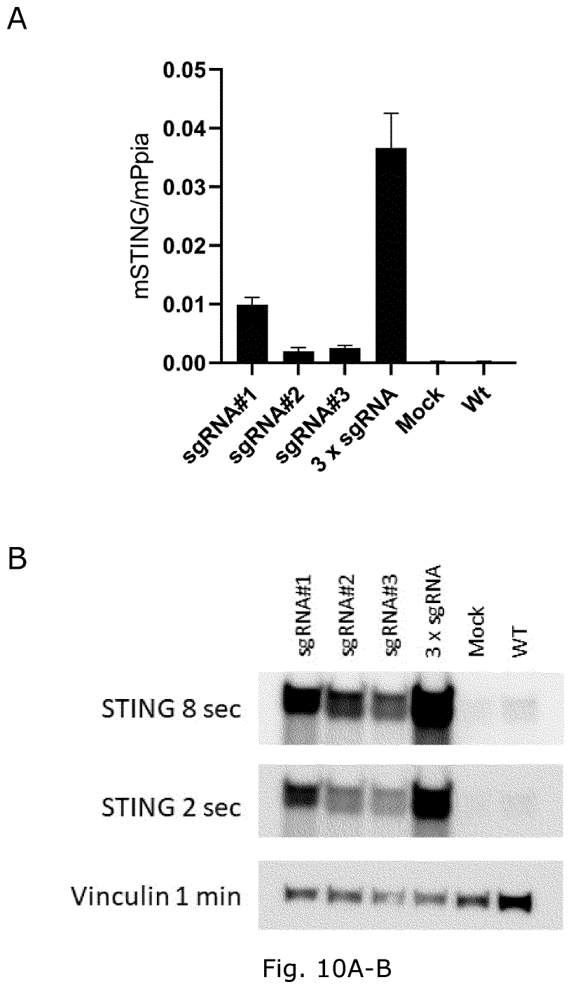

A series of proprietary sgRNAs targeting various regions surrounding the transcriptional start site of the murine STING gene was designed (SEQ ID NO's: 6-8). These sgRNAs were combined with dCas9-VPR mRNA and electroporated into the cell line CMT-167. After 48 hours, cells were lysed and STING expression was determined by qPCR (FIG. 10A) and immunoblotting (FIG. 10B).

### Conclusion

In summary, these data demonstrate that murine STING expression can be found genetically re-expressed using a group of sgRNAs combined with dCas9-VPR expression within murine cancer cells.

Verification in a murine system is important for later in vivo experiments in mice.

### Example 11—Reactivating Genetically Silenced STING in 4T1 and CMT-167 Over Time Using the CRISPRa Technology

### Aim of Study

Explore the possibility to reactivate genetically silenced STING in 4T1 and CMT-167 over time using the CRISPRa technology.

### Materials and Methods

### CRISPR Activation of STING In Vitro

Murine cell line CMT-167 and 4T1 were purchased from ATCC were all cultured at 37° C. with 5% CO₂ and under humidified conditions. CMT-167 were cultured in RPMI-1640 (Sigma-Aldrich, Cat#: R8758) supplemented with 10% fetal bovine serum (Sigma-Aldrich, Cat#: F9665), 1% penicillin/streptomycin (Gibco, Cat#: 15140122), and 1% L-glutamine (Thermo Fisher Scientific, Cat#: 25030024). 4T1 cells were cultured in Dulbecco's modified eagle's medium (Sigma-Aldrich, Cat#: D6429) supplemented with 10% fetal bovine serum (Sigma-Aldrich, Cat#: F9665), 1% penicillin/streptomycin (Gibco, Cat#: 15140122), 1% L-glutamine (Thermo Fisher Scientific, Cat#: 25030024).

Cell lines were routinely tested for mycoplasma using the service of Eurofins Genomics.

The sequence of the DNA binding part of the 3 sgRNAs were the following:

#6:
(SEQ ID NO: 6)
CCAGUCUCAGGAUGGUUGAG,
#7:
(SEQ ID NO: 7)
AGGUACUAGAAUUAAAUGAA,
#8:
(SEQ ID NO: 8)
GAAACAGGAUUAGAAGCCUU.

sgRNAs were purchased from Synthego and apart from the mRNA+sgRNA-electroporated cells, a wt control and a mock control was included which went through the same treatment except no sgRNA and dCas9-VPR mRNA was added. For each sample 500,000 cells were resuspended in PBS, spun down at 300 xg for 5 minutes at room temperature (RT) and resuspended in 17 μL opti-MEM (Gibco, Cat#: 31985070). 1.9 μL of dCas9-VPR mRNA (Horizon Discovery, Cat#: CAS12211) and 1 μg of each sgRNA (1 μg of each sgRNA in the sample with all 3 sgRNAs) were added. The cell suspensions were added to an electroporation strip and electroporated on a Lonza 4D nucleofector using pulse code CM138. Afterwards, the electroporation strip containing the cells were left for 3 minutes on the bench top to allow cells to reform. The samples were diluted in preheated culture media and 10,000-60,000 cells were seeded in a 24-well plate and left for either 2 days, 3 days, 4 days, 5 days or 6 days before harvest.

### Western Blotting

Cells were lysed in RIPA buffer (Thermo Fisher Scientific, Cat#: 89901) supplemented with 10 mM NaF (VWR, Cat#: J60251.AE), pierce protease and phosphatase inhibitor (Thermo Fisher Scientific, Cat#: A32961), protease inhibitor cocktail (Roche, Cat#: 11873580001), and benzonase (Millipore, Cat#: E1014-25KU) in a volume of 1 μL/mL lysis buffer. Cell lysates were diluted 1:1 with Laemmli Lysis Buffer (Sigma-Aldrich, Cat#: 38733). The lysates were denatured at 95° C. for 5 minutes and 15 μL lysate were separated on a 10% Criterion Precast protein gel (Bio-Rad, Cat#: 5671035) with MOPS SDS running buffer (Invitrogen, Cat#: NP0001). The proteins we transferred to a 0.2 μm PVDF membrane (Bio-Rad, Cat#: 1704157) using a Trans-Blot Turbo Transfer System (Bio-Rad). A TBS wash buffer (fisher scientific, Cat#: BP2471-500) supplemented with 0.05% Tween-20 (Sigma-Aldrich, Cat#: P1379) (TBS-T) was used for washing the membranes. Membranes were blocked in 5% skim-milk (Sigma-Aldrich, Cat#: 70166) in TBS-T.

Membranes were incubated ON at 4° C. with the following primary antibodies in 5% BSA (Roche, Cat#: 10735086001) in TBS-T: anti-STING (rodent) (Cell Signaling Technologies, clone: D1V5L, Cat#: 50494S) diluted 1:1000 and anti-vinculin (Sigma Life Sciences, Cat#: V9131) diluted 1:10000. The following day, the membranes were incubated for 1 h at RT with the following secondary antibodies diluted 1:7500 in 5% skim milk in TBS-T: Peroxidase AffiniPure F(ab′) 2 Fragment Donkey Anti-Mouse IgG (Jackson Immuno Research, Cat#: 715-036-150), Peroxidase AffiniPure F(ab′) 2 Fragment Donkey Anti-Mouse IgG (Jackson Immuno Research, Cat#: 711-036-152). The membranes were developed using Clarity Western ECL Substrate (Bio-Rad, Cat#: 1705060).

### Real-Time PCR

RNA from cells was purified using RNeasy Mini kit (Qiagen, Cat#: 74104) according to manufacturer's guidelines. cDNA was synthesized using iScript cDNA Synthesis Kit (Bio-Rad, Cat#: 1708891) according to manufacturer's guidelines using 200 ng of RNA. Real-time PCR was carried out using 1 μL of 10 ng/μL cDNA in a total volume of 10 μL KAPA SYBR FAST (Roche, Cat#: KK4611) and the following targets and primers:

| | |
|---|---|
| Target | Primer SEQ ID NO |

| | | |
|---|---|---|
| TMEM173 | Forward | SEQ ID NO: 23 |
| (STING) | Reverse | SEQ ID NO: 24 |
| ppia | Forward | SEQ ID NO: 25 |
| | Reverse | SEQ ID NO: 26 |

Samples were run in both biological and technical duplicates on a 384 well plate (Hounisen, Cat#: 72.1985.202). The analysis was performed using the LightCycler 480 software version LCS480 1.5.1.62(Roche).

### Results

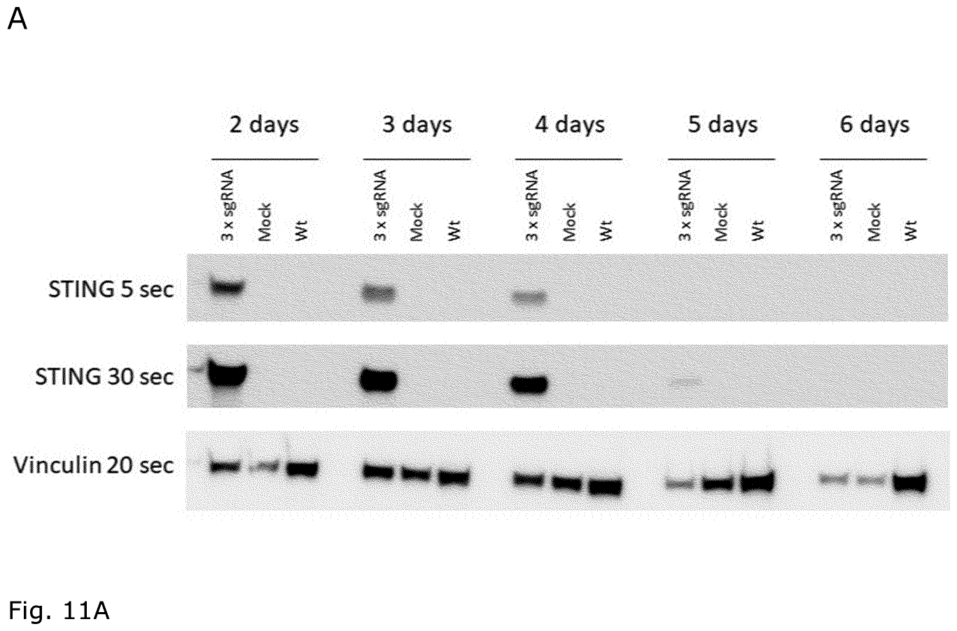

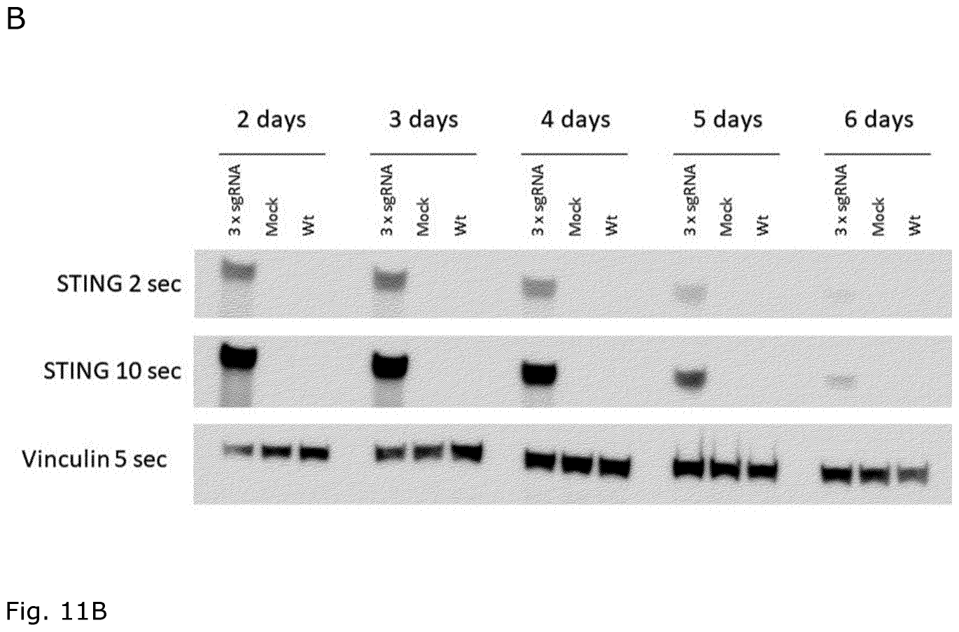

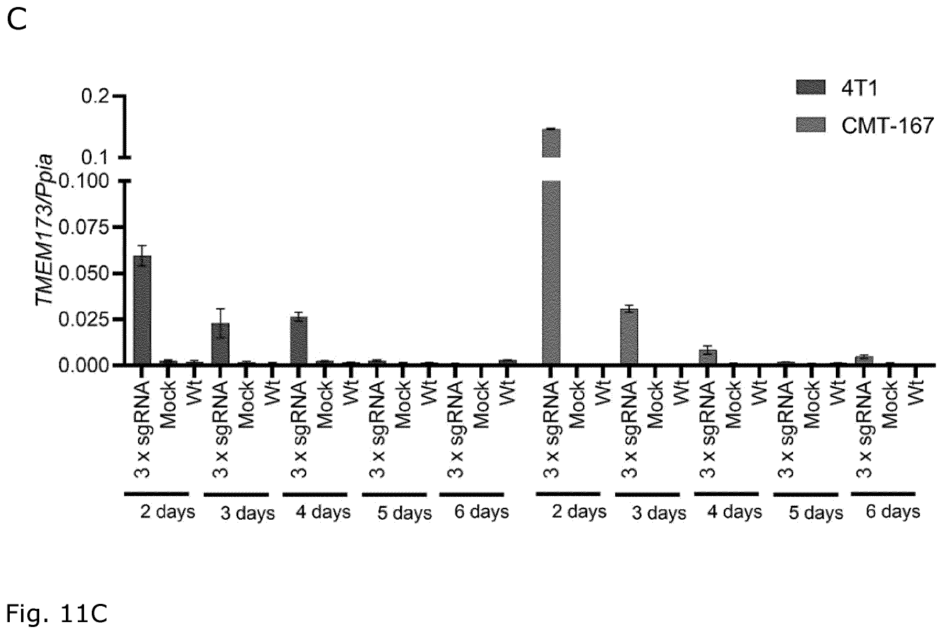

A series of proprietary sgRNAs targeting various regions surrounding the transcriptional start site of the murine STING gene was designed (SEQ ID NO's: 6-8). These sgRNAs were combined with dCas9-VPR mRNA and electroporated into the cell line 4T1 (FIG. 11A) or CMT-167 (FIG. 11B). After 48, 72, 96, 120 and 144 hours, each cell population was lysed and STING expression was determined by immunoblotting (FIG. 11A+B) or by qPCR (FIG. 11C). The results demonstrated that STING protein expression was rapidly induced in both cell lines after 24 hrs and continued to be expressed for up to 144 hours. However, gene expression disappeared after 96 hours post electroporation.

### Conclusion

In summary, these data demonstrate that murine STING expression can be genetically re-expressed using a combination of sgRNAs with dCas9-VPR in numerous murine cancer cell lines without endogenous STING expression.

### Example 12—Immune Activation Potential in Murine Cancer Cell Lines 4T1 and CMT-167 After STING-Targeted CRISPRa Reactivation

### Aim of Study

Explore the immune activation potential in murine cancer cell lines 4T1 and CMT-167 after STING-targeted CRISPRa reactivation.

### Materials and Methods

### CRISPR ACTIVATIOn of STING In Vitro

Murine cell line CMT-167 and 4T1 were purchased from ATCC were all cultured at 37° C. with 5% CO₂ and under humidified conditions. CMT-167 were cultured in RPMI-1640 (Sigma-Aldrich, Cat#: R8758) supplemented with 10% fetal bovine serum (Sigma-Aldrich, Cat#: F9665), 1% penicillin/streptomycin (Gibco, Cat#: 15140122), and 1% L-glutamine (Thermo Fisher Scientific, Cat#: 25030024). 4T1 cells were cultured in Dulbecco's modified eagle's medium (Sigma-Aldrich, Cat#: D6429) supplemented with 10% fetal bovine serum (Sigma-Aldrich, Cat#: F9665), 1% penicillin/streptomycin (Gibco, Cat#: 15140122), 1% L-glutamine (Thermo Fisher Scientific, Cat#: 25030024).

Cell lines were routinely tested for mycoplasma using the service of Eurofins Genomics.

The sequence of the sgRNA was the following: #6: CCAGUCUCAGGAUGGUUGAG (SEQ ID NO: 6). The sgRNA was purchased from Synthego and apart from the mRNA+sgRNA-electroporated cells, a wt control and a mock control was included which went through the same treatment except no sgRNA and dCas9-VPR mRNA was added.

For each sample 500,000 cells were resuspended in PBS, spun down at 300 xG for 5 minutes at room temperature (RT) and resuspended in 17 μL opti-MEM (Gibco, Cat#: 31985070). 1.9 μL of dCas9-VPR mRNA and 1 μg of sgRNA was added. The cell suspensions were added to an electroporation strip and electroporated on a Lonza 4D nucleofector using pulse code CM138. Afterwards, the electroporation strip containing the cells were left for 3 minutes on the bench top to allow cells to reform. The samples were diluted in preheated culture media and 40,000 wt cells and 80,000 mock/STING+cells were seeded in a 24-well plate one day prior to stimulation to allow cells to adhere.

### In Vitro Stimulation of Cell Culture

Cells were stimulated by transfection using Lipofectamine 2000 transfection reagent (Invitrogen, Cat#: 11668019). Cells were transfected with 1 μg/mL HT-DNA (Sigma-Aldrich, Cat#: D6898). An untreated control and a lipofectamine 2000 control were also included. All samples were run in biological triplicates. HT-DNA was mixed with lipofectamine 2000 in a ratio of 1:1 according to manufacturer's guidelines. For stimulation, the growth media was removed, and fresh growth media containing the stimulation and transfection agent was added and left at 37° C. with 5% CO₂ and under humidified conditions for 24 hrs. Supernatants were harvested and used for cytokine detection assays.

### Enzyme-Linked Immunosorbent Assay (ELISA)

Supernatants were analyzed for cytokines using sandwich ELISA for the following targets: IL-6 (RnD Systems, Cat#: DY406), CCL5 (RnD Systems, Cat#: DY478), and CXCL10 (RnD Systems, Cat#: DY466). All were carried out according to manufacturer's guidelines.

### Results

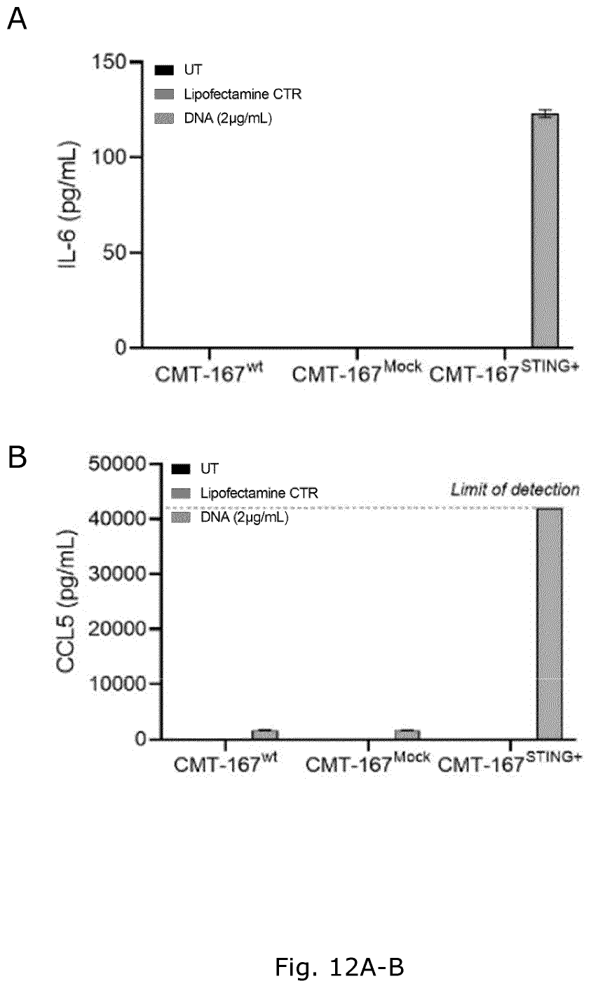

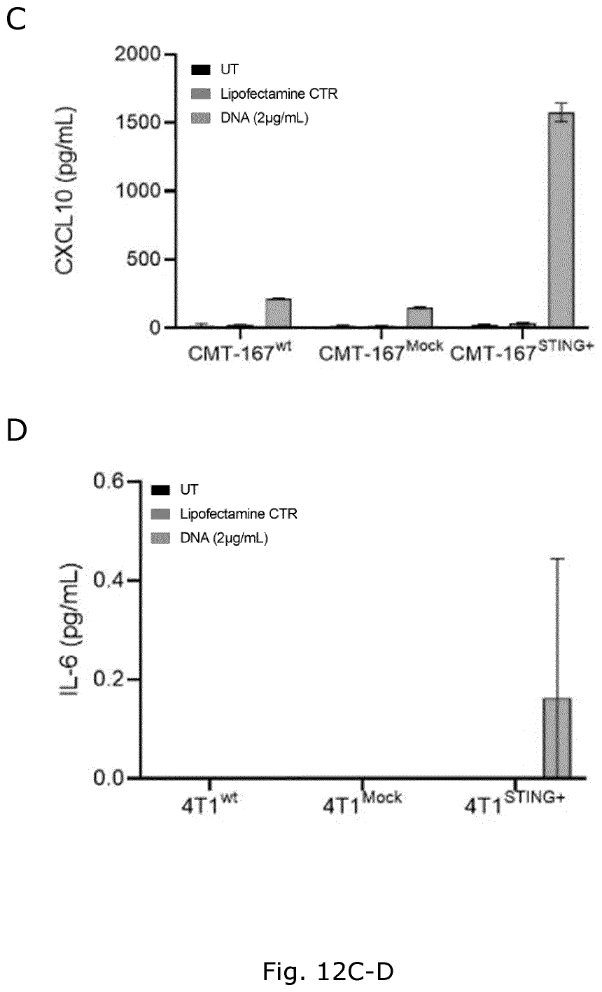

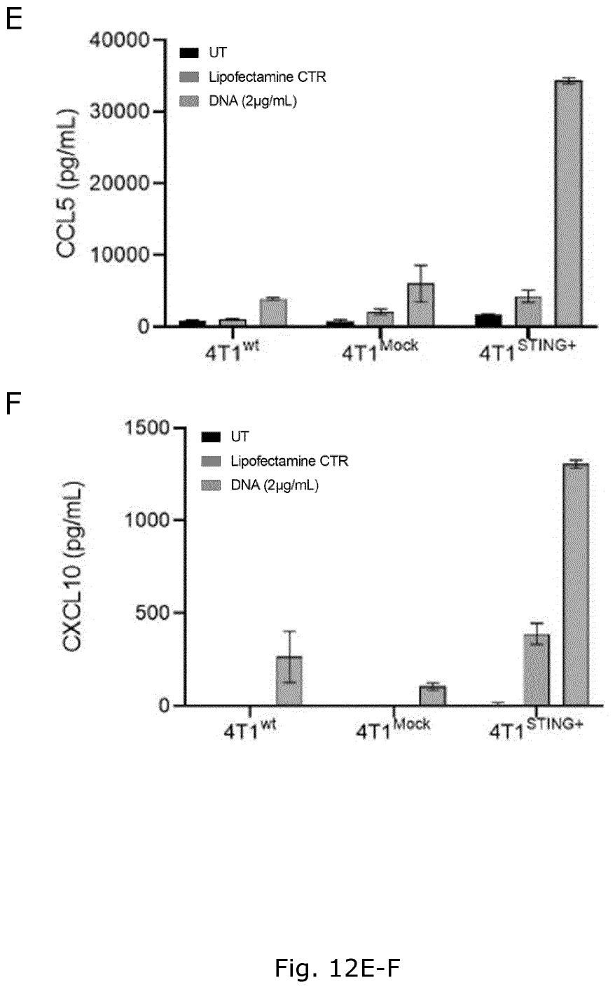

Here, we repeated an electroporation of 4T1 and CMT-167 with dCas9-VPR mRNA (SEQ ID NO: 29). As controls, cell samples were electroporated without the addition of sgRNA and dCas9-VPR (mock) as well as a cell sample left untreated (WT). After 24 hrs each cell conditions were stimulated with HT-DNA. The level of STING activation was determined after additional 24 hrs using murine IL6, CCL5, and CXLC10 ELISA for CMT-167 (FIG. 12A-C) and 4T1 (FIG. 12D-F). The results demonstrate that wildtype (wt) and mock treated cells were either none-responsive or gave a low response to HT-DNA stimulation, whereas CRISPRa reactivation of STING in both CMT-167 and 4T1 resulted in strong cytokine secretion levels of CCL5 and CXL10 and to a lesser extend IL6.

### Conclusion

In summary, these data demonstrate that STING re-expression using a combination of sgRNA and dCas9-VPR mRNA can sensitize murine cancer cells to respond to STING activation and become immunologically active.

### Example 13—Broad Immune Activation After STING-Targeted CRISPRa Reactivation Induced by Endogenous DNA Sensing and cGAMP Production

### Aim of Study

Explore the immune activation potential in murine cancer cell lines 4T1 after STING-targeted CRISPRa reactivation.

### Materials and Methods

### Cell Lines

Murine cell line 4T1 were purchased from ATCC and cultured in Roswell Park Memorial Institute (RPMI) 1640 medium (Sigma-Aldrich, Cat#: R8758-500ml) supplemented with 10% fetal bovine serum (Sigma-Aldrich, Cat#: F9665), 1% penicillin/streptomycin (Gibco, Cat#: 15140122), 1% L-glutamine (Thermo Fisher Scientific, Cat#: 25030024). YUMM1.G was cultured in Dulbecco's modified eagle's medium (Sigma-Aldrich, Cat#: D6429) supplemented with 10% fetal bovine serum (Sigma-Aldrich, Cat#: F9665), 1% penicillin/streptomycin (Gibco, Cat#: 15140122), 1% L-glutamine (Thermo Fisher Scientific, Cat#: 25030024). Cell lines were routinely tested for mycoplasma using the service of Eurofins Genomics.

### IVT

The dCas9-VPR mRNA IVT plasmid was purified using the Qiagen Plasmid Midiprep kit (Qiagen, Cat#: 12945). Following purification, the plasmid was linearized using Anza™ (Thermo Fisher Scientific, Cat#: IVGN0334). The linearized product was then purified using GeneJet PCR Purification kit (ThermoFisher Scientific, Cat#: K0701), according to manufacturer's guidelines. The quality of the linearization process was verified by running 500 ng of linearized plasmid together with an uncut plasmid control on a 0.5% agarose gel (Thermo Fisher Scientific, Cat#: 16500-100), made using TAE buffer (Thermo Fisher, Cat#: B49). To synthesize the mRNA, the MEGAscript T7 transcription kit was used (Life, Cat#: AM1334). NTPs (ATP, CTP, GTP) (5 mM) along with pseudo UTP (5 mM) (Nordic Biosite, Cat#: 333-B7972-100UL), with 4 mM CleanCap® Reagent AG (Tebu-bio, Cat#: N-7113-5), and nuclease-free water was added. Next, the 10x Reaction buffer, and 1 μg of linearized product was added. Finally, the enzyme was added. The reaction mixture was incubated 2-4 hours at 37° C., before being stopped with the addition of 1 μl included Turbo DNase. The final mRNA product was purified using the Monarch RNA Cleanup kit (New England Biolabs, Cat#: NEB-T2050L).

### CRISPR Activation of STING In Vitro

The sequence of the DNA binding part of the 3 sgRNAs were the following:

#6:
(SEQ ID NO: 6)
CCAGUCUCAGGAUGGUUGAG,
#7:
(SEQ ID NO: 7)
AGGUACUAGAAUUAAAUGAA,
#8:
(SEQ ID NO: 8)
GAAACAGGAUUAGAAGCCUU.

sgRNAs were purchased from Synthego and apart from the mRNA+sgRNA-electroporated cells, a mock control was included which went through the same treatment except no sgRNA was added.

For each sample 500,000 cells were resuspended in PBS, spun down at 300xG for 5 minutes at room temperature (RT) and resuspended in 17 μL opti-MEM (Gibco, Cat#: 31985070). 1,9 μg of dCas9-VPR mRNA and 1 μg of each sgRNA was added. The cell suspensions were added to an electroporation strip and electroporated on a Lonza 4D nucleofector using pulse code CM138. Afterwards, the electroporation strip containing the cells were left for 3 minutes on the bench top to allow cells to reform. The samples were diluted in preheated culture media and seeded in a 24-well plate one day prior to stimulation to allow cells to adhere.

### CGAMP ELISA

In a 24-well plate, 30.000-100.000 cells of either 4T1 or YUMM1.G were seeded out in 0.5 ml growth medium. The following day, cells were either transfected with 2 μg/ml HT-DNA (Sigma-Aldrich, Cat#: D6898) or kept untreated, and cultured for additional 24 hours in culture medium either with or without 50 μM STF-1084, (an ENPP1 inhibitor). Supernatants were collected and stored for downstream analysis. The cell pellets were lysed in M-PER lysis buffer (Thermo scientific, Cat#: 78501) for 5 minutes on a tilting table. The levels of intracellular (cell pellet) and extracellular (supernatants) 2′3′-cGAMP were evaluated using a 2′3-cGAMP ELISA Kit (Cayman Chemical, Cat#: 501700) according to manufacturer's protocol.

### Micronuclei Analysis by Confocal Microscopy

Cells were seeded on 12 mm glass coverslips (#630-2190, VWR) at a density of 20.000 cells per well in a 24-well plate. Cells were incubated for 48 h in an incubator at 37 C with 5% Co2 and then fixed in 2% formaldehyde in PBS for 10 min. at RT. Subsequently, Hoechst33342 (#62249, Thermo Fischer Scientific) was added to the mixture for the last 10 min. of secondary staining at a concentration of 10 μg/mL. Cells were washed once in PBS and coverslips were mounted onto microscope slides (#48311-703, VWR) with a drop of mounting medium (ProLong™ Gold Antifade Mountant, #P36930, Thermo Fischer Scientific). Images were acquired on an inverted Zeiss LSM800 laser scanning confocal microscope using a Plan Apochromat 40x/1.4 NA oil objective. Diode laser 405 was used at 0.15% power, detector gain was 650 V and pixel time was 1.52 μs with bidirectional scanning and averaging of 2. Z-stacks were acquired with slice thickness of 0.19 μm. For quantification of micronuclei in cells, images were batch processed using Fiji macro (run(“Z Project . . . ”, “projection=[Max Intensity]”); run(“Enhance Contrast”, “saturated=0.80”); run(“Apply LUT”);). Nuclei and micronuclei were then manually counted using the Cell Counter plugin in Fiji and represented as micronuclei per cell. A minimum of 10 images were counted per group. Statistics and graphs were done in GraphPad Prism version 9.3.1 for Windows (GraphPad Software, San Diego).

### RNAseq Preparation and Analysis

Cells were lysed in RA1 buffer (Macherey-Nagel, Cat#: 740961) supplemented with 20 mM DTT (Sigma-Aldrich, Cat#: 10197777001). RNA was isolated using the Machery-Nagel Nucleospin RNA kit (Machery-Nagel, Cat#: 740955.250). The quality and purity of the RNA was then ensured using the BioTek Take3 microvolume plate (Agilent, Cat#: TAKE3-SN). Afterwards, the RNA samples were diluted using Nuclease-free water (Synthego), to a final concentration of 500 ng in 10 μl. The samples were frozen, before being sent for RNAseq at Novogene. The mRNA library preparation was done through poly A enrichment. Sequencing was performed on the Illumina platform, which sequenced PE150, the output of which was 6 G raw data per sample.

### Western Blotting

Cells were lysed in RIPA buffer (Thermo Fisher Scientific, Cat#: 89901) supplemented with 10 mM NaF (VWR, Cat#: J60251.AE), pierce protease and phosphatase inhibitor (Thermo Fisher Scientific, Cat#: A32961), protease inhibitor cocktail (Roche, Cat#: 11873580001), and benzonase (Millipore, Cat#: E1014-25KU) in a volume of 1 μL/mL lysis buffer. Cell lysates were diluted 1:1 with Laemmli Lysis Buffer (Sigma-Aldrich, Cat#: 38733). The lysates were denatured at 95° C. for 5 minutes and 15 μL lysate were separated on a 4-20% Criterion Precast protein gel (Bio-Rad, Cat#: 5671095) with MOPS SDS running buffer (Invitrogen, Cat#: NP0001). The proteins we transferred to a 0.2 μm PVDF membrane (Bio-Rad, Cat#: 1704157) using a Trans-Blot Turbo Transfer System (Bio-Rad). A TBS wash buffer (fisher scientific, Cat#: BP2471-500) supplemented with 0.05% Tween-20 (Sigma-Aldrich, Cat#: P1379) (TBS-T) was used for washing the membranes. Membranes were blocked in 5% skim-milk (Sigma-Aldrich, Cat#: 70166) in TBS-T for one hour.

The membranes were then washed 3×5 minutes in TBS-T, before being incubated ON at 4° C., on a rocking table, with the following primary antibodies in 5% BSA (Roche, Cat#: 10735086001) in TBS-T: anti-STING (rodent) (Cell

Signaling Technologies, clone: D1V5L, Cat#: 50494S) diluted 1:1000 and anti-vinculin (Sigma Life Sciences, Cat#: V9131) diluted 1:10000. Anti-phosphor-STING (Rodent) (Cell Signaling Technologies, clone: D8F4W, Cat#: 72971) diluted 1:1000. Anti-STAT1 (Rodent) (Cell Signaling Technologies, clone: D1K9Y, Cat#: 14994) diluted 1:1000. Anti-ISG15 (Rodent) (Thermo Fisher Scientific, clone: 1H9L21, Cat#: 703132) diluted 1:5000.

The following day, the membranes were washed 3×5 minutes in TBS-T before being incubated for 1 h at RT with the following secondary antibodies diluted 1:7500 in 5% skim milk in TBS-T: Peroxidase AffiniPure F(ab′)₂ Fragment Donkey

Anti-Mouse IgG (Jackson Immuno Research, Cat#: 715-036-150), Peroxidase AffiniPure F(ab′)₂ Fragment Donkey Anti-Mouse IgG (Jackson Immuno Research, Cat#: 711-036-152). The membranes were developed using Clarity Western ECL Substrate (Bio-Rad, Cat#: 1705060), or SuperSignal™ West Femto Maximum Sensitivity Substrate (Thermo Fisher Scientific, Cat#: 34095

### Results

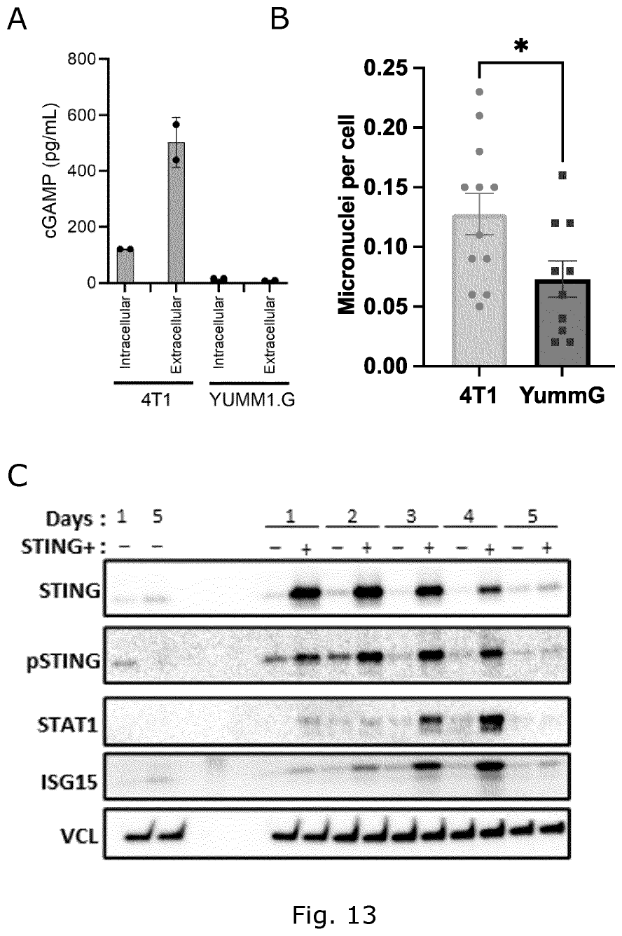

First, we evaluated the level of endogenous cGAMP production in 4T1 and the control murine cancer cell line YUMM1.G, which has an active STING pathway. The intracellular and extracellular levels of cGAMP was measured by ELISA. The results demonstrate that 4T1 cells have a significant production of endogenous cGAMP in absence of stimulating the cells (FIG. 13A). This could be explained by the higher degree of micronuclei formations in 4T1 (FIG. 13B) supporting cytosolic DNA sensing by cGAS.

Next we repeated electroporation of 4T1 with dCas9-VPR mRNA (SEQ ID NO: 29) and sgRNA targeting STING. As controls, cell samples were electroporated without the addition of dCas9-VPR mRNA or sgRNA as well as a cell sample left untreated (WT). Cells were harvest for western blot analysis each day for up to 5 days after electronation. The results from this kinetic experiment demonstrate that STING protein expression was rapidly elevated after 1 day and started to decline after 4 days. In conjunction to elevated STING protein expression we observed a delayed activation of the cGAS-STING pathway, measured by phosphorylation of STING and elevated expression of the interferon stimulated genes (ISGs) of STAT1 and ISG15, which peaked at day 3 and 4 post CRISPRa of STING (FIG. 13C).

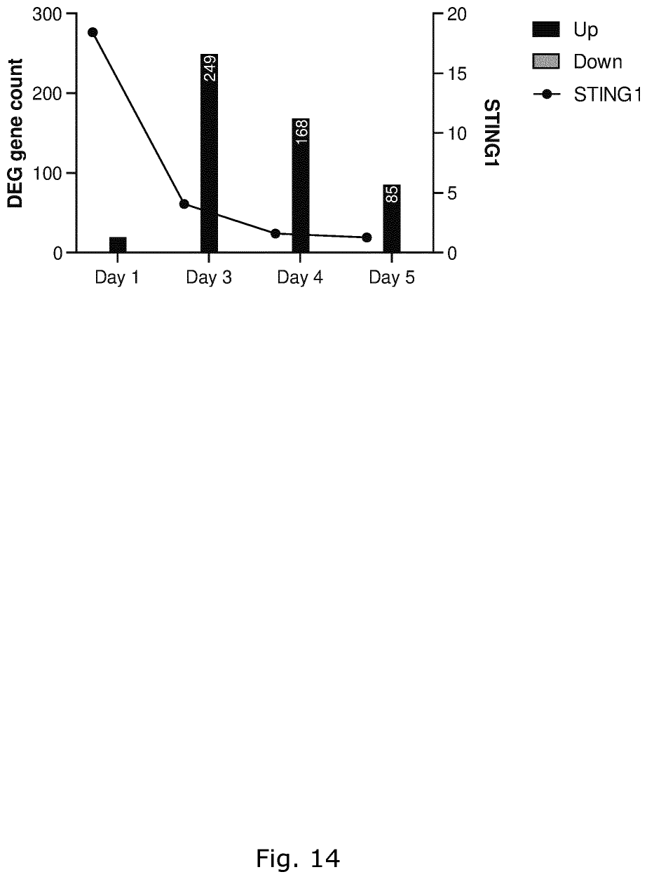

Next, we repeated this experiment in biological triplicates and collected cells after Day 1, 3, 4 and 5 post CRISPRa STING, and conducted RNA sequencing and bioinformatic analysis. Investigation of differential expressed genes (DEGs) demonstrate that re-expression of STING in 4T1 support a major upregulation of genes at day 3, which subsequently decrease over the following days. In parallel, STING mRNA expression is highest at day 1 and decrease to undetectable levels at day 5 (FIG. 14). A GO-enrichment analysis of molecular pathways on the samples from day 3, demonstrates that the upregulated genes correlate with increased activation of innate immunological pathways such as “response to virus”, “defense response to virus”, “cellular response to interferon-beta”, “cytokine-mediated signaling pathway” and many more (Data not shown).

### Conclusion

In summary, these data demonstrate that STING re-expression using a combination of sgRNA and dCas9-VPR mRNA can activate the STING pathway to respond to endogenous levels of cGAMP within cells, which is produced as a consequence of DNA instability and/or increased micronuclei formation leading to DNA cytosolic accumulation and activation of cGAS. This cGAS activation produce CGAMP which binds to re-expressed STING protein leading to elevated expression of hundreds of genes known to be involved in anti-tumoral, anti-viral and pro-inflammatory pathways.

### Example 14—Reactivating Genetically Silenced STING in 4T1 Using the CRISPRa Technology Combined with Lipid Nano Particles

### Aim of Study

Explore the possibility to reactivate genetically silenced STING in cancer cells by delivery of mRNA CRISPRa components with a clinical applicable lipid nano particle delivery system.

### Materials and Methods

Lipid nanoparticles (LNPs) encapsulating STING sgRNA were prepared on the NanoAssemblr Ignite (Precision Nanosystems) using sgRNA dissolved in 0,1 M sodium acetate buffer and a lipid formulation composed of 1,2-dimyristoyl-rac-glycero-3-methoxypolyethylene glycol-2000 (DMG-PEG2000, Avanti Polar Lipids), 1,2-Distearoyl-sn-glycero-3-phosphocholin (DSPC, Avanti Polar Lipids), Cholesterol (Sigma Aldrich) and SM102 (BOC Sciences) in the molar ratio 1:10:39:50 in abolute EtOH. sgRNA-containing LNPs are formulated at an N/P ratio of 10, using a flow rate ratio (lipid: mRNA) of 1:4 and a total flow rate of 5 ml/min. After preparation, the LNPs are dialyzed twice against 1L of 150 mM NaCl O/N at 4° C. and sterile filtered using a 0.2 um cellulose acetate filter (Avantec). Final RNA concentration is determined using the Quant-it RiboGreen RNA Assay Kit (Thermo Fisher Scientific), and lipid content is determined by high-pressure reverse phase chromatography (UV absorbance at 210 nm) using a Dionex Ultimate 3000 HPLC system (Thermo Fisher Scientific) on a Ascentis C18 column (Sigma Aldrich).

The sequence of the DNA binding part of the 3 sgRNAs were the following:

#6:
(SEQ ID NO: 6)
CCAGUCUCAGGAUGGUUGAG,
#7:
(SEQ ID NO: 7)
AGGUACUAGAAUUAAAUGAA,
#8:
(SEQ ID NO: 8)
GAAACAGGAUUAGAAGCCUU.

sgRNAs were purchased from Synthego.

The dCas9-VRP mRNA was produced from IVT plasmid (see example 13 method section).

### LNP-CRISPR Activation of STING In Vitro

Murine cell line 4T1 was purchased from ATCC and cultured at 37° C. with 5% CO₂ and under humidified conditions using Dulbecco's modified eagle's medium (Sigma-Aldrich, Cat#: D6429) supplemented with 10% fetal bovine serum (Sigma-Aldrich, Cat#: F9665), 1% penicillin/streptomycin (Gibco, Cat#: 15140122), 1% L-glutamine (Thermo Fisher Scientific, Cat#: 25030024). Cell lines were routinely tested for mycoplasma using the service of Eurofins Genomics.

For each sample 100,000 cells were seeded in a 24-well plate prior to treatment with LNPs packed with sgRNA or with dCas9 mRNA. A series of RNA concentrations were evaluated including 500, 1000 and 2000 ng, per 200.000 cells.

### Western Blotting

Cells were lysed in RIPA buffer (Thermo Fisher Scientific, Cat#: 89901) supplemented with 10 mM NaF (VWR, Cat#: J60251.AE), pierce protease and phosphatase inhibitor (Thermo Fisher Scientific, Cat#: A32961), protease inhibitor cocktail (Roche, Cat#: 11873580001), and benzonase (Millipore, Cat#: E1014-25KU) in a volume of 1 μL/mL lysis buffer. Cell lysates were diluted 1:1 with Laemmli Lysis Buffer (Sigma-Aldrich, Cat#: 38733). The lysates were denatured at 95° C. for 5 minutes and 15 μL lysate were separated on a 10% Criterion Precast protein gel (Bio-Rad, Cat#: 5671035) with MOPS SDS running buffer (Invitrogen, Cat#: NP0001). The proteins we transferred to a 0.2 μm PVDF membrane (Bio-Rad, Cat#: 1704157) using a Trans-Blot Turbo Transfer System (Bio-Rad). A TBS wash buffer (fisher scientific, Cat#: BP2471-500) supplemented with 0.05% Tween-20 (Sigma-Aldrich, Cat#: P1379) (TBS-T) was used for washing the membranes. Membranes were blocked in 5% skim-milk (Sigma-Aldrich, Cat#: 70166) in TBS-T.

Membranes were incubated ON at 4° C. with the following primary antibodies in 5% BSA (Roche, Cat#: 10735086001) in TBS-T: anti-STING (rodent) (Cell Signaling Technologies, clone: D1V5L, Cat#: 50494S) diluted 1:1000 and anti-vinculin (Sigma Life Sciences, Cat#: V9131) diluted 1:10000. The following day, the membranes were incubated for 1 h at RT with the following secondary antibodies diluted 1:7500 in 5% skim milk in TBS-T: Peroxidase AffiniPure F(ab′)₂ Fragment Donkey Anti-Mouse IgG (Jackson Immuno Research, Cat#: 715-036-150), Peroxidase AffiniPure F(ab′)₂ Fragment Donkey Anti-Mouse IgG (Jackson Immuno Research, Cat#: 711-036-152). The membranes were developed using Clarity Western ECL Substrate (Bio-Rad, Cat#: 1705060).

### Real-Time PCR

Cells were lysed in RA1 buffer (Macherey-Nagel, Cat#: 740961) supplemented with 20 mM DTT (Sigma-Aldrich, Cat#: 10197777001). RNA was isolated using the Machery-Nagel Nucleospin RNA kit (Machery-Nagel, Cat#: 740955.250). The quality and purity of the RNA was then ensured using the BioTek Take3 microvolume plate (Agilent, Cat#: TAKE3-SN). The cDNA was synthesized using iScript cDNA Synthesis Kit (Bio-Rad, Cat#: 1708891) according to manufacturer's guidelines using 200-1000 ng of RNA. After synthesis, the cDNA was diluted for a final concentration of 10 ng/μL.

Real-time PCR was carried out using 1 μL of 10 ng/μL cDNA, 5 μL KAPA SYBR FAST (Roche, Cat#: KK4611), 0.05 μL Precision Blue™ Real-Time PCR Dye (Bio-Rad, Cat#: 1725555), nucleas-free water, and primers (10 μM, for a final volume of 10 μL per well. The primers used and their targets are as follows:

| | |
|---|---|
| Target | Primer SEQ ID NO |

| | | |
|---|---|---|
| TMEM173 | Forward | SEQ ID NO: 23 |
| (STING) | Reverse | SEQ ID NO: 24 |
| ppia | Forward | SEQ ID NO: 25 |
| | Reverse | SEQ ID NO: 26 |

Samples were run in both biological and technical duplicates on a 384 well plate (Hounisen, Cat#: 72.1985.202). The analysis was performed using the LightCycler 480 software version LCS480 1.5.1.62(Roche).

### Results

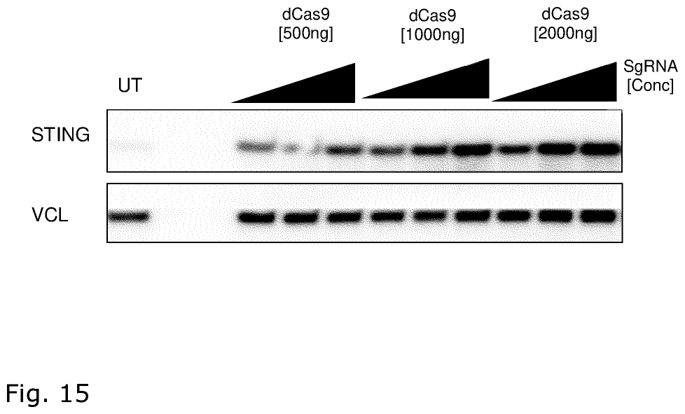

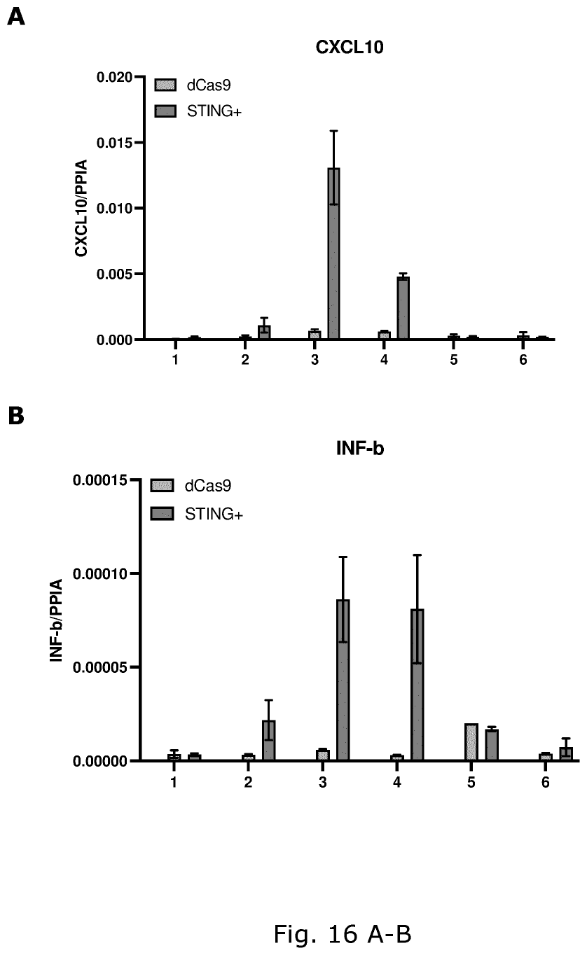

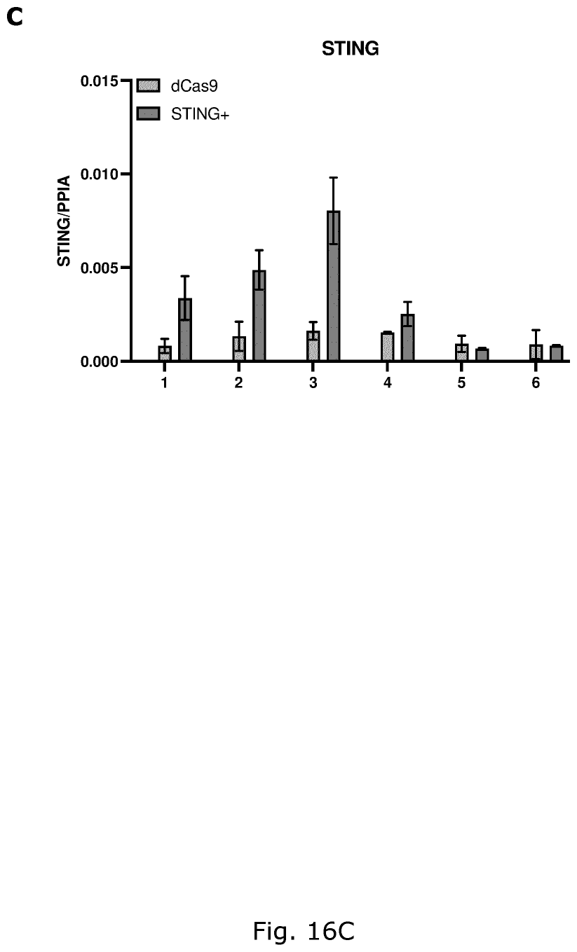

Following LNP delivery of sgRNA as well as dCas9 mRNA to 4T1 cells in culture, we observed a clear concentration dependent reactivation of STING protein levels (FIG. 15A). Based on these data we next treated 4T1 cells with the highest concentration of RNA (2000 ng LNP-sgRNA and 2000 ng LNP-dCas9 mRNA) and harvest the cells after 1, 2, 3, 4, 5, and 6 days post LNP treatment. QPCR analysis of STING mRNA expression demonstrated rapid gene expression after day 1 which peaked at day 3 and then reached baseline levels at day 6 (FIG. 16A). The induction of CXCL10 and IFNb genes peaked at day 3 following a rapid decline after day 4 (FIG. 16B+C).

### Conclusion

In summary, these data demonstrate that re-expression of STING in cancer cells can be achieved by delivery of sgRNA and dCas9 mRNA by lipid nanoparticles. The effect and kinetic of STING re-expression and endogenous STING activation followed the same pattern as we had observed for CRISPRa done by electroporation.

### Sequence Listing

TABLE 1
Overview of sequences used

| SEQ ID | | |
|---|---|---|
| NO | Name | Sequence 5′ => 3′ |
| 1 | #1 | GGUUCCUACCUCCCUUCCUG |
| 2 | #2 | UUUACUGGUGCUGGGAAGGA |
| 3 | #3 | GAGUGUGUGGAGUCCUGCUC |
| 4 | #4 | CAGAAACCGGCAGGCUCUCU |
| 5 | #5 | GAGGAGGGGCACAGAGGAAU |
| 6 | #6 | CCAGUCUCAGGAUGGUUGAG |
| 7 | #7 | AGGUACUAGAAUUAAAUGAA |
| 8 | #8 | GAAACAGGAUUAGAAGCCUU |
| 9 | TMEM173 (STING) | CATGGGCTGGCATGGTCATA |
| | Human-FW | |
| 10 | TMEM173 (STING) | AACCCGATCCTTGATGCCAG |
| | Human-REV | |
| 11 | IL-6-FW | TGTGAAAGCAGCAAAGAGGC |
| 12 | IL-6-REV | ACCTCAAACTCCAAAAGACCAGT |
| 13 | CXCL10-FW | GAACCTCCAGTCTCAGCACC |
| 14 | CXCL10-RV | GCAGGTACAGCGTACAGTTCT |
| 15 | CCL5-FW | AGGATCAAGACAGCACGTGG |
| 16 | CCL5-RV | TACTCCTTGATGTGGGCACG |
| 17 | IFNB1-FW | TGCTCTCCTGTTGTGCTTCT |
| 18 | IFNB1-RV | AGCCTCCCATTCAATTGCCA |
| 19 | IFNL1-FW | GGTGACTTTGGTGCTAGGCT |
| 20 | IFNL1-RV | GGAAGACAGGAGAGCTGCAA |
| 21 | EIF2B2-FW | CAGAGAGGGCAGGAGGATGA |
| 22 | EIF2B2-RV | CCTGCTGATCACTCTCGTCG |
| 23 | TMEM173 (STING) | GGGCCTTCAGAGCTTGACTC |
| | Murine-FW | |
| 24 | TMEM173 (STING) | TTGAACATTCGGATCCGGGC |
| | Murine-REV | |
| 25 | ppia-FW | ATGGTCAACCCCACCGTG |
| 26 | ppia-RV | TTTCTGCTGTCTTTGGAACTTTGTC |
| 27 | STING gene | See sequence listing |
| 28 | 5′ STING fragment | See sequence listing and Table 2 below |
| 29 | dCas9-VPR | See sequence listing |
| 30 | dCas9 | See sequence listing |
| 31 | VPR | See sequence listing |
| 32 | sgRNA- scaffold | GUUUUAGAGC UAGAAAUAGC AAGUUAAAAU |
| | | AAGGCUAGUC CGUUAUCAAC UUGAAAAAGU |
| | | GGCACCGAGU CGGUGCUUUU |

TABLE 2
SEQ ID NO: 28 details.

SEQ ID NO: 28
CTGCAACTTCCACCTCTCAGGTTCCAGCGATTCTCCTGCCTCAGCCTCCCAAGTAGCTAAGATT
ACAAGCGCCCACCACCACGCCTGGCTAATTTTTGTTTTTAGTAGAGATGGGGTTTCACCATGTT
GCTCAGGCTGGTCTTGAACTCCTGACCTCAAGTGATCCACCCATCTCGGCCTCCCAAAGCGCTG
GGATTACAGGCATGAGCCACTGTGCCAGGCCTGCAATTACTTTTGCTCCTACCTAATATCATCC
CCACAACCGCCTTCTGGGCAGAAACCGGCAGGCTCTCTTGGAGAAGTCACAGGCGTGGCCATTT
CCTGCAAAGAGCCAAACCCCCATTCCTCTGTGCCCCTCCTCTCCCACCAAGTGCTTTATAAAAA
TAGCTCTTGTTACCGGAAATAACTGTTCATTTTTCACTCCTCCCTCCTAGGTCACACTTTTCAG
AAAAAGAATCTGCATCCTGGAAACCAGAAGAAAAATATGAGACGGGGAATCATCGTGTGATGTG
TGTGCTGCCTTTGGCTGAGTGTGTGGAGTCCTGCTCAGGTGTTAGGTACAGTGTGTTTGATCGT
GGTGGCTTGAGGGGAACCCGCTGTTCAGAGCTGTGACTGCGGTGAGTGTTTCTAAACACCCTTG
GTTTGGGGGTAGCAAAGGGCAATGGAATGGAGGCTTTCTCAAACCTCACCCCTGACCCCAGGAC
TCAGGCCCAGCTCATCAGGGCTTTGAGGGAAGGTTCCTACCTCCCTTCCTGAGGAACAGGAAAT
ACCCTCCTTCCCAGCACCAGTAAAGCTGCGGTTTGGAGAAACGCCAGGGCTAGAGTGTTGTGGA
GAAACCAATCGTTGTTAACATCTCATTTTCAGGCTGCACTCAGAGAAGCTGCCCTTGGCTGCTC
GTAGCGCCGGGCCTTCTCTCCTCGTCATCATCCAGAGCAGCCAGTGTCCGGGAGGCAGAAGGTA
GGCTCAAGATCAGCCTGGCAGAACGCCAAACCTAGGGCCCCTGGCACCCAGAGGCGAGGGGGTG
CCTGCTGGCTGCCCTGTCCCCACTCCCTGAGCTCTGTTTTCCACTTTGTTGACTAAGGTCCTCC
CTGGGGTGGGTTCCGGGGACAGGGGAACCCAGGTCCCCAAGGGTTCTTGGTTGGGTACGGCTGC
ACAGGACAGCTTCAA
The DNA sequence surrounding the transcriptional start site of the human
STING gene (same sequence as in SEQ ID NO 28). Target sequence (+ or −
strand) for the CRISPRa sgRNAs #1-5 (SEQ ID NO's: 1-5) are underlined and in
the order 5′ to 3′: #4, #5, #3, #1 and #2. PAM sites in bold and italic font.
Exon 1 and 2 are marked in grey. See also FIG. 17.

1. A CRISPR activation (CRISPRa) composition or a CRISPRa combination comprising
a)
an RNA molecule encoding a deactivated RNA-guided endonuclease, such as dCas, preferably dCas9; or
a deactivated RNA-guided endonuclease, such as dCas, preferably dCas9; and
b) a guide RNA complementary to a part of STING DNA; and
c) a transcriptional activator
encoded by the RNA molecule encoding the deactivated RNA-guided endonuclease; or
fused to the deactivated RNA-guided endonuclease;
wherein the guide RNA binds to STING DNA within SEQ ID NO: 28 or the complementary sequence of SEQ ID NO: 28.

2. The CRISPRa composition or CRISPRa combination according to claim 1, comprising an RNA molecule encoding an RNA-guided protein.

3. The CRISPRa composition or CRISPRa combination according to claim 1, wherein the guide RNA comprises one or more oligonucleotide sequences selected from the group consisting of SEQ ID NO's: 1-5.

4. The CRISPRa composition or CRISPRa combination according to claim 1, the guide RNA binds to STING DNA within position 270-300 of SEQ ID NO: 28 or the complementary sequence of SEQ ID NO: 28.

5. The CRISPRa composition or CRISPRa combination according to claim 1, wherein the transcriptional activator is selected from the group consisting of RTA, p65, VP16, HSF1, MyoD1, VP64, VP160, repeats of VP16, CBP, p300, and combinations thereof.

6. The CRISPRa composition or CRISPRa combination according to claim 1, wherein the 3′ end of the hybridizing part of the guide RNA binds 1-10 nucleotides upstream of the 5′-end of the PAM sequence.

7. The CRISPRa composition or CRISPRa combination according to claim 1, wherein the composition or combination is located in/on LNPs.

8. The CRISPRa composition or CRISPRa combination according to according to claim 1, wherein
the RNA molecule encoding a deactivated RNA-guided endonuclease or the RNA-guided endonuclease is positioned in a first LNP; and
the guide RNA comprising a sequence complementary to a part of STING DNA) is positioned in a second LNP.

9. A method of preventing, treating and/or ameliorating a cancer in a subject in need thereof, said method comprising administering a CRISPRa composition or CRISPRa combination subject in need thereof;
said CRISPRa composition or CRISPRa combination comprising
a)
an RNA molecule encoding a deactivated RNA-guided endonuclease; or
a deactivated RNA-guided endonuclease; and
b) a guide RNA complementary to a part of STING DNA; and
c) a transcriptional activator
encoded by the RNA molecule encoding the deactivated RNA-guided endonuclease; or
fused to the deactivated RNA-guided endonuclease;
wherein the guide RNA binds to STING DNA within SEQ ID NO: 28 or the complementary sequence of SEQ ID NO: 28.

10. (canceled)

11. (canceled)

12. The CRISPRa composition or CRISPRa combination according to claim 1, wherein the deactivated RNA-guided endonuclease is a dCas9.

13. (canceled)

14. (canceled)

15. (canceled)

16. (canceled)

17. The CRISPRa composition or CRISPRa combination according to claim 1, wherein the guide RNA comprises SEQ ID NO: 4.

18. The method according to claim 9, wherein the guide RNA comprises one or more oligonucleotide sequences selected from the group consisting of SEQ ID NO's: 1-5.

19. The method according to claim 9, wherein the CRISPRa composition or CRISPRa combination comprises an RNA molecule encoding an RNA-guided protein.

20. The method according to claim 9, wherein said subject has undergone cancer therapy or is undergoing cancer therapy, or who is scheduled for cancer therapy-of said cancer, with a different therapy.

21. The method according to claim 9, wherein the cancer is associated with no or low STING activity

22. The method according to claim 9, wherein the cancer is a metastatic cancer, a refractory cancer, and/or recurrent cancers.

23. The method according to claim 9, wherein the cancer is a solid cancer.

24. The method according to claim 9, wherein said cancer is a haematological cancer.

25. The method according to claim 9, wherein the guide RNA binds to STING DNA within position 270-300 of SEQ ID NO: 28 or the complementary sequence of SEQ ID NO: 28.

26. The method according to claim 9, wherein the guide RNA comprises SEQ ID NO: 4.
